SEQUENCE LISTING (19 sequences)

SEQ ID NO: 1 - DNA, 601 bp, Homo sapiens
  source 1..601: mol_type = other DNA
ccctcatgctagcaatcctttctcagtcagctctgccattacagaggatggttgtaacaaattttgtcctctgaaactaagcaaaatatatcaatttctcacagctgacagagccaaaaggtggaaaggcttgatataagtaaaacaatggaatgcttagctgcaggcctagaaaggaccctttaattgccaggctctgtcaccatatcaagcgtggtagggttcggggctgaagcatacttacgaagacacacaaggcagtagctggtaccctcacttctttaccagaaccaggatgagyatccacattgtcctgggggactgggaaggaagagacagagcgtctcctaagaaataacataaagacaaatattagacaggattgcagaggtttactgctcatcaaattgttagaaaggactccaagacgaccttgcttaagcagactgcctctgttgatagcctgtccttctagattcttcaaactacagaacaattcacaaaaaaatcaaaagcaccctcactcaaatgagaaagagagccagcacatgccttactctcctgcacactaacctgtggactccaaaacccaacaatgaaa

SEQ ID NO: 2 - DNA, 646 bp, Homo sapiens
  source 1..646: mol_type = other DNA
aagatttattgggggtaagaagagctaatgcctatgaaagataaaggagaaaggagcagaagtacggagagaaaagacagctttcagactgcagtccagatctaactctgggacgcaagagagggaaggataattctgttgaaagagcatcagactgtgatgcggctgtaagagtgtctcaacgagcccagtggggagttccagccaaagattgcccaggagaagagtcgcactttgggcagaaatggacaggcccgagcaaccctgccatgttctgtcattggctgggggccacccaggaggcaacatggtctgacttgaatggtgtggatccgaggctgcagcctgtcagctgtctgcactccatgcaacaggtcctttgaatggcatgtgttcgtggctgccataaactgcagcctgccttttagccttacctcctttgctcttgctctctgacccygtgttctggcaacactggcctgactacacgccgtatcacatacaaccaactgcccataccaaccccaaccctgacctttgctcactcagttctttctgcctggaatgccactccttcccagttatacccagcaaaatcctaatgtgctttcaaggcccaacttaaaaatcatcttctctggttgac

SEQ ID NO: 3 - DNA, 601 bp, Homo sapiens
  source 1..601: mol_type = other DNA
atctttatggaaagatgttcattattcactatattgaattttataatgttaaactctgcttttctttgcataaactatcagagattataatactaaaatcttaaatagttaaattataagtaaattttgtgttttcccatatataaaagtgatatatttaatgaatgctagtaatcttaaactggttatataattttatactacaatgagtaccttcgagaaagcttatggtataagaaatactatttccaaaacatttttgttgcacatttttggtattagactcatcattccaatgacrtctggattatgggaagaaaggagcctgactcttatgatggaataaccacaaatcagagaggagtcacaatagcagctcttggtgcagactgtataccgatagtttttgcagatccagtcaaaaaagcatgtggggttgctcacgctggtaagtatacttaattaaacatttagaattttactcattttgttgtgcaggaaaaattgtaatttctttctgatggacatggcaaacagttaattacattacactgtggtaagtgtaatgatgggagaggcggcatagttttattatagggac

SEQ ID NO: 4 - DNA, 634 bp, Homo sapiens
  source 1..634: mol_type = other DNA
cccgccctgccaaggaatgtcacgagcctcaagcagatttgctcttagatatttctttttaatcgagtctaacagtgattgttgagccgtccttcatttttttttttttttgtaagatgcctctgtcaagaaggagccacgagttggctttcatggaatgggtgctcttggtggccaaatagaaataaaacctggctgtctggtcctgatccactcacagaagtggcgtaaacaccttatttatgatctgggacattcaacaccatcttataaaagattcatagaatacacattcacacacccacaaaaatcaaragagcagattttccaaaaataggtgcaagaaagatccaacttccaggataaaattttagaaccaacatttccattagcaaaagcggtcttgtctaaaactctaccgaccgcgctaaaatcccattcaaaacagagctctggttacgtagatcgtgatcaataatgagaaactgtgaggcgtccccgtgggatctgacacttcctaggggagcttctctccactggtaataaagatataattaggaatttcaactacctgtgattttgccaacttacggcatttgttgagaatttcggtccctgtgtttatgcacacacc

SEQ ID NO: 5 - DNA, 801 bp, Homo sapiens
  source 1..801: mol_type = other DNA
ctagaacatatatgccaatgggtagtgatacacaactgtcactggtaaagttgcataccaattcacaatcctattctcatacccaagttactactacacattgctcttgagtcagtagctgcatttgccaccctggctcaaaatatcagccatcacatttagtaaattctaacatacaaagcaaattagcactgatttatcagatttaccattgggtggggtgaggaggaggaggatgctggctgacttagcccagtcccttaagagctctgttccagagaatatacttgtgctcttcctccatggcccagcatatgcacagggctccagtcatttgcctccagcagggggctcttccacactcattcacagtcagctggattccaatctctgctcaagtrtgagcaatgtagatccagccccctggttaatgtattcatcactgttcaagcccagtctctttcagatgttgagacagtggccctaactctgtgtggctggcccagagctgtgcacctaccctcactttcataccacattaatttcagatccttattgtcatgggtttcccaactacttttttttcttcaggggaaacctccacaatgtagtttctaatatgttgaattcatactccagaaagtgtcctgtagaataatgtcttactgaaaacggccatcacagccaggagtccttaactatgttctttgatacccttagttacagtttgttgtcatgttcttcacatcttgtgtgaagattgttcaagtattggccaaaggatatgtcactatctaaaat

SEQ ID NO: 6 - DNA, 401 bp, Homo sapiens
  source 1..401: mol_type = other DNA
atagctgtaacactgtgtgcaggtatctggggtttctgtcgtgaccacgtggcaggagctgctgccactgctgtctgatgctcgccccacagtggaaggagatgctaaattccgttacgcattagaggtcagtgaaaaggaagatgcagtttgttcccgtccaggcacaaggactcttgaatttgtccatagttaagaacrgctcatccaggagcagagcgagaggccgggctgcgcgtcctcatctcctctcccagccttcgcatcctcctggctgcctcgcgtttcctccacgggcctggctgaacgcacacacaggcctgggggagactgcagagacacatcttcagccacatcttctgtaaaacagtcactatgggatgacggtgactggacagtgg

SEQ ID NO: 7 - DNA, 601 bp, Homo sapiens
  source 1..601: mol_type = other DNA
acaatccaaacagggtagtggttacccctgaggagaaaactaggtaggagcatggcgggagaggaagattgagaggtctaaggcaactttatctactttccaaaatgttttaaaataagaatgctttattatttgtgtcatttttaaattaataaagtaggcatgccattctgaaattccacgtctaaatggcacaaaagtgtaagataggcaccacagttgcttatacaacattactctccaaagattatattcccaaacgctttacctattccctcttagaaaagacttccagagtctytcagtcttcatcttggcctgtatttttaacccattaggcatcctcttggggtaacttcagcaggctccataggtacaacaaccttcacgtgatctttaaattagctccataatagtgataatgaggcaggaaccttgagataaaaagcagatattacaggttccactttcgcttctagcactaatgattacatggaaagtcgctcaacccaagttatagcttccacattcatatggaaggaataacaacaagaattatacctcactcctactcagggacatgcaggtaggaaccacaaca

SEQ ID NO: 8 - DNA, 401 bp, Homo sapiens
  source 1..401: mol_type = other DNA
aacggatcatcaactccaggatactcacttttgctaaattctcaatactttgtaaagatatcatgtccaggtgaacgttaaaataatcttaccttggggacctcataagatgtggttctagtcaaacacacttattccaggatatatttcagagtgactccaaattcccatctgttgctagaaccaatcatttctgccgtkccaaaaaattatcgtagctcagaagacctatgttaaaaaggccaaaaaaaaaaaaggaatcaattagcattttaatgttaaaatgaaactaatttccagtagcaaaataatggtttttaaccatgacacctgcagtttcaggcaagtggcagacagccaaacatatccatagttgtaaggtgtcctactattagggagga

SEQ ID NO: 9 - DNA, 201 bp, Homo sapiens
  source 1..201: mol_type = other DNA
agctgatgtctgtctcatgggcaaatacaacaaatactgacatagatggcctcaagagaagaaaaagtggcaaaacaccttattgactgcttgttcgcagrctaaggttgtgacgagaagtacaagagaatgaatttggccagctatctggagagtagggtacctgtgaagcccaggcctctaaatggggtcctctcacat

SEQ ID NO: 10 - DNA, 401 bp, Homo sapiens
  source 1..401: mol_type = other DNA
cttcatttcaaaaaaagttttggcatttacggaaggcttcttaaacttcttccatggtttgacatcattgactcagataaaggcctaggaagccattcctcctattcaggatctaaaaaagcagcagttcacatttataacgcattacaaccagaacttgaggcctaagaaatttctatgtcgagtccacagtttcaaacrcaaattcactcatcaaggctttgttcaggcatcagggaaacatcctggcagcaagtatacctatataaggaaatattaggtcacgatcccataactccaacacacaagagatgcaccacacactttaggagacttccaaagcagaaaaacacaagagactagggagaatgacctctcttcctagtttatgccttcaaggt

SEQ ID NO: 11 - DNA, 401 bp, Homo sapiens
  source 1..401: mol_type = other DNA
accataaatcaataagaaaaaacaaatgatgaacaattcagtatgagaataagcaaagaatataaacaaatcattcaaaaaggaggaaattaaagcttcagagaagcatgtaaaggtaatatccaactccactaacaagaagggaaaaattgaaatgaaaattaaaactgtaatacagcagtattcttatgagaaagaacrtagaatttctgtgatacactgtgctttcaaaggcatatagacaaagtactcatgcattgttagaagaagggtaatttaacaatatctatcaatgttaaaaattgcttacatcttttgatgcagcaaattgatgtatatatgtttccctataaacatatatacaaaggtattcttcccatcattgtttggaatagccccaa

SEQ ID NO: 12 - DNA, 1001 bp, Homo sapiens
  source 1..1001: mol_type = other DNA
agactcaatcacatgaggactctaacttcagcagctattccaaatttgaccttataactaaagaaaatcaacacattattttgcacctgaaatgcagttcttgacgcggtgaattccccctccacccatcccccgcatacctggtaatgaacatcatcagaagaagttgattttaagctaggtagtcaagtacacatcttcattgtttaattgcagaactctatcaaatctccaactcaaactatcctatgtgggctttccacaaatgacagcatcattctggctcattcgattcatcaacaatttgctgatcagaccaactcaaaggtaaatagcaattattttagaaattgtaataagggacacgttgcagagagcaggaaataaatctcaccaaaaagtgagggcctgctaccttggtgaagtttctacatatggtttggaagatatcctggtcccatctgaagttgctagttgcgttgccacagctccatgcaggartgcctatgatggacaaaagtgaagaaaaatcttcccggtgggcagagttttgagcagagcaccaagtaattcattttttaatggaggtagaaattgatcaatacatagatctatgttgactgatgagcagtaacaaattggctgcataatgggtaacttggaaaggatatagctaagagattttaggtaagaaattttgctgaataaatctaaggataaacttctttcagttggcacagaatgtgaagatatatgtgcccaacataaatgttaacctaggaaaaacctattagtaattaggcggataagatactctgttctctagacataattcccccttttccccaaccactctgtccttgcaaatgtggttcatgagcaacatggctatgacagcagagattaagttatgcatggactcactaaataatacctattgctgagtgcctaacttgccaacagcaggttggtactcactgggtgcacactcagctggcaca

SEQ ID NO: 13 - DNA, 809 bp, Homo sapiens
  source 1..809: mol_type = other DNA
tggaaatcttgggaaaatttgcatgtaattagtactcccttccagctctccgaagtataaaatacaatacattagggcttgtaacactaatatcgctgagaacacagctaatgtgggaagtctaattcgccactaatatgcagatgccaagctagttggtgcttgctttcacytgttttggctaattgttttcggtgtgatttagaaactccataccttatgatgcagttgtacctgatgcaacaactcaccagtgctggactgtgggaggtcacatcatttagactgcaaattcagaagtgactttcgaggaaatttggtcccgttcccaagcaataaagccgtcgcatcataatcagcttagaacaatttgccaatgatagttttctgtgttgcgccatcagagattctgattcagcaagtcctcgttggatctcatacatctcaattaaaaatcaacaatagatcgggcgtggtggctcacgcctgtctgtcatcccagcactttgagaggccaagccaggtggatcaggagttccagaccagcctggccaacatggtgaaaccccatctctgctaaaagtacaaaaattagccaggcgtggtggtgcatgcctgtagtcccagctactggggaggctgaggcaggagaataacttgagtccgggaggcggaggttgcaatgagccaagatcgtgcaactgtactccagccctgggtgacagagcaagaatctgttctcaaataattaaataactaagatattgattaattaacaattcatgtctttttgaatgcgaccagaaaat

SEQ ID NO: 14 - DNA, 1008 bp, Homo sapiens
  source 1..1008: mol_type = other DNA
aaaaaaaaaaaaaaaaaaagaaagaaagaaagaaagaaaggtaggaaggacaaaggaactcctgcctcctgtgtcctcccacagcatatcactggggggccctgtcaaatgaggggcactaggttgagaactataaaacaatctgttgatttatgcatttaaaacctcaagacttttttttttttttttttctggagatggagtttcgctcttgttgcccaggctggagtgcaatggtgtgatctcggctcactgtaacctccaccttccgggttcaagccattcttttgcctcagcctccccagtagctggaattataagtaagcaccaccatgcccggctaattttttatatttttagtagagacgaggtttttccatgttagtcaggctggtctcgaactcccgacctcaggtggtcctcccacctcagcctcccaaagtgctggaattacaggcatgagccaccacgcctggttggacttttgtctcaaatgaaattgttaaaggaacccacattttacaaaatatgtggtatgtggtggaaaaaacttccttccatgagtctcttttccagggtatatgggagataaggaggggaagaccagggcttttaggcccggctcttcctggtgctgagatactggaagactgccttgtaaagcatctagggatgtaaattcaggaaaagttctttaagcattagaccaaagctgagtgagctgggagattatggatctcgcatgtagacctgataacgggcatcggtaggatcttaatccttggagagccaggtgcctccctctgcccacatgatgtgcatgcaagtaaaaaagaaacatggaactaaattatctcccagtttttcctcctccgcagtcccctccagctctatccttcctttctaccaagagcagagccacttagaggacaaggtactcaycacgtctggctcaggtccaatgctgtgaagaggtaccagatacaagagacaggtgggactggctca

SEQ ID NO: 15 - DNA, 601 bp, Homo sapiens
  source 1..601: mol_type = other DNA
agatgcggtattttatgaaatacaagaagaaaaatgctctaaaacccataatcataaacacagcatatggaaaaatgaaagcgctagttttgagttagaagcatcttttatctacttccactccctccactttctggtcaatattagatacataatcagagaaatgcttaaatttcaccaaaggcttgcttgagttggaagcttgattttttttttagaagaaattccccaccatccattctactgcaaagattttctttctcaagtgctttcacctgtcaaaagccctctgtccagatayttagcagatccctccactctctcattctgcaaggcagagggaagaggaggctacattgagtgagtgtctaccatgaaccaggctcagacacagacatctggcatctcatgccggcctctcagaggggctgcgaggacagtgggctgcccacgggctctcgggttcagagaggcagtgagcctggctgaagggcaccagctaggaaatggcagagccagattctgcacatccccaaagcccaagacacctccctacctcccagacttctccatttgagtatatgcgccctgacgaaaagca

SEQ ID NO: 16 - DNA, 501 bp, Homo sapiens
  source 1..501: mol_type = other DNA
ctatctgcatttcagagagggcttttcagttttgttataaaacctcattacttttttattacagacttagtggagccttctctgttgaattgaatcatatgattcactattagctggacatttaatactatatcgtctttatattgttcaggattcttctcagttcttttatttgcactttggaaatgtctcaggagaagaaattgaaagatgatagttctttcaaataattattccctgtcccaggaagrtagtgttttaaagacaattatttaaacaaaatatactaccattttctaattctttttgatttaagctccctacaataatgaatgagaagaatgagaattaagcggtttagaaaacaatgtggtttttggcaaaatcaaaatatttttaaaaatcctttaagatcatcagtgcataccacatgttttgtgaagcttttgtagaatctctgaggtgtccacctctcttattgtacttattcacatctcttcc

SEQ ID NO: 17 - DNA, 601 bp, Homo sapiens
  source 1..601: mol_type = other DNA
aaaccgctttacaacacttacaactttatttaacagcagaaagctcaaagctgccatgcagagggaagctatgtagactctggaagccttgaagcttaggagcataggaattacagctctgtctggttggcttaaccacatccaggcatggaggcaaggaaaaaaaaaaaaactcaaagctttgtttggaggatggtactaggaaagaactaggaaactagtgagagaaggtaccaagctatgcctttgtccacccactattcaatttacaaatatttcttggtcctatatcaggcactcyattgggtactaggaataaatacgcagatgaatgaggcatggctctttccccaaaaaggacttatatgtgctcaaaatgtgttacagaaatcccatgaggattcttggatattccaagagggtcaaatttcattgctctgtttttcatacctgggactgggctttagtagactctctctggtttcctgaagcttcataccccacatctgccctactgtgagagtccaaccccagggctctagctggcttctctcaactgttctacagttttctaggccacagcttgagatgtgacagaaag

SEQ ID NO: 18 - DNA, 801 bp, Homo sapiens
  source 1..801: mol_type = other DNA
tttgataaggaagtgagtggtgtgaaatccaggctcaagaacaaaagccctacaagcctgtacccaacggggccactggcctagaatggtaataaatagaattgggtgacaaccttgaaggtcacccgtggttcagctctggaggtgtccttgtttgtactcactttgagtaaccatcaacttataaatagcctcaggccaattgcccagtgatgattttgactgtcgcttgccaggggcctgggcttgtggtttggttgtgagtctgtcatggtctctgtgacctcagaggaggaggaaggaagatgatgatggatgcttctagtgatgcaagcaacaaacatttttggacaatgtgcccaataccattctctgtactctgcatgagttaacccttcagkcttcaaacaacgctttgaggtaggtgaggagactgagcactaaaaaggtgaatgcggagccagtgtgagcacaaaaggcctggttccctgctagaccgtgcctcctcttggctggagctctatggaaagagttggaaccagggagccttggttaaaatatcagctccatcccttatgaccttgggcaacttacaaatctccaaatctgtattttcaccttttaactagaataatgtatgtcaattatagggtgttaggattaaatgagaaaatgtacataaaacttttggggcattgtctggcactgggtaagtactcaatgaaaaaaggtaatggttttttaacttgtttaatgtttctaagccttaggttttccacttataaaatgaaagcgtcatta

SEQ ID NO: 19 - DNA, 1001 bp, Homo sapiens
  source 1..1001: mol_type = other DNA
ctggttgacagcgcaagactcccttccccccccaaaaaaatctcctaatcttctgtgtctgtgtggcaagccatgaactccttctcagaagaatgtttttaaatgcattaaataaagcatgaggtggggagagttccaaaggaagccagtgatactaaaacatacagaattatcaaacagtgtttatgcttctgttataagaatagatgggctactttcacatattaaacagcaagagctagcagcaagtctagtgaactgggcacttggaagcagtgatgagcatagcagtatctctggatagctgtaacactgtgtgcaggtatctggggtttctgtcgtgaccacgtggcaggagctgctgccactgctgtctgatgctcgccccacagtggaaggagatgctaaattccgttacgcattagaggtcagtgaaaaggaagatgcagtttgttcccgtccaggcacaaggactcttgaatttgtccatagttaagaacrgctcatccaggagcagagcgagaggccgggctgcgcgtcctcatctcctctcccagccttcgcatcctcctggctgcctcgcgtttcctccacgggcctggctgaacgcacacacaggcctgggggagactgcagagacacatcttcagccacatcttctgtaaaacagtcactatgggatgacggtgactggacagtggctccgtgcagtcggtgtcttttgcaaagttttccaccgtgtgcatcctctgcagccaactccagctgagggagaacggaccctcggtgagttggggctgcacagcaggtatggcctgctgccttcaccccacacctgctatcaaatgaatcagccccctttccctacaaccccaagaaaagcctcaggacccatcaggggaagaagaagctgcccatatagagagagggaagggatactgctcctcattccccacacggcatagcccaggtgtagaaagtggccaacatccatttaggaa

(19) United States
(12) Patent Application Publication
(10) Pub. No.: US 2025/0059605 A1
(43) Pub. Date: Feb. 20, 2025

(54) METHODS OF DETERMINING RESPONSIVENESS TO ANTI-TNF ALPHA THERAPY IN INFLAMMATORY BOWEL DISEASE

(71) Applicant: CEDARS-SINAI MEDICAL CENTER, Los Angeles, CA (US)

(72) Inventors: Jerome I. Rotter, Los Angeles, CA (US); Marla Dubinsky, Los Angeles, CA (US); Stephan R. Targan, Santa Monica, CA (US); Kent D. Taylor, Ventura, CA (US)

(21) Appl. No.: 18/795,930

(22) Filed: Aug. 6, 2024

Related U.S. Application Data
(63) Continuation of application No. 17/551,028, filed on Dec. 14, 2021, now Pat. No. 12,084,722.
Application No. 17/551,028 is a continuation of application No. 16/025,769, filed on Jul. 2, 2018, now Pat. No. 11,236,393.
Application No. 16/025,769 is a continuation of application No. 13/130,998, filed on May 24, 2011, now abandoned.
(60) Provisional application No. 61/182,552, filed on May 29, 2009.
(60) Provisional application No. 61/142,307, filed on Jan. 2, 2009.
(60) Provisional application No. 61/118,290, filed on Nov. 26, 2008.

(51) Int. Cl. C12Q 1/6883

(52) U.S. Cl. CPC C12Q 1/6883; C12Q 2600/106; C12Q 2600/156; C12Q 2600/172

(57) ABSTRACT

The present invention relates to methods of prognosing responsiveness to anti-TNFα therapy by determining the presence or absence of risk factors in the individual. In one embodiment, the risk factors are genetic markers, serological markers and/or clinical phenotypes associated with non-responsiveness to treatment with anti-TNFα therapy in an individual diagnosed with IBD.

### CROSS-REFERENCE TO RELATED APPLICATIONS

This application is a continuation of U.S. application Ser. No. 17/551,028, filed Dec. 14, 2021, which is a continuation of U.S. application Ser. No. 16/025,769 filed Jul. 2, 2018, which is a continuation of U.S. application Ser. No. 13/130,998 filed May 24, 2011, which is a National Phase of International Application PCT/US09/65928, filed Nov. 25, 2009, which claims the benefit of U.S. Provisional Application No. 61/118,290, filed Nov. 26, 2008, U.S. Provisional Application No. 61/142,307, filed Jan. 2, 2009, and U.S. Provisional Application No. 61/182,552, filed May 29, 2009.

### GOVERNMENT RIGHTS

This invention was made with government support under Contract Nos. DK046763, and DK56928 awarded by the National Institutes of Health. The government has certain rights in the invention.

### SEQUENCE LISTING

The instant application contains a Sequence Listing which has been submitted electronically and is hereby incorporated by reference in its entirety. Said copy, created on Aug. 2, 2024, is named 56684-707.303.xml and is 29,296 bytes in size.

### FIELD OF THE INVENTION

The invention relates generally to the field of inflammatory bowel disease and, more specifically, to genetic methods for diagnosing, prognosing, and treating inflammatory bowel disease.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Natural history observations in both early and later onset inflammatory bowel disease (IBD) have prompted the increasing use of anti-TNFα therapy for IBD patients. Various past studies have demonstrated that infliximab has the potential to be effective for the induction and maintenance response and remission in some CD patients. However, the clinical trial data for all anti-TNFα therapies among adult CD patients report that 40% of patients do not respond to the induction phase (primary non-responder) and that approximately 40% of those patients who do enter the maintenance phase of the trial lose response over time. The pediatric REACH trial, for example, reported that close to 90% of children responded to induction, suggesting a more robust acute response to anti-TNFα therapy in children as compared to adults with CD. This primary response outcome did not, however, require children to have weaned corticosteroids to meet response criteria. This would be a more clinically robust outcome definition given that the importance of steroid sparing in the induction and maintenance phase of these therapies. Moreover, approximately 40% of children, like their adult counterparts, who entered the maintenance phase lost response and were no longer in remission and off steroids at 12 months. More studies are needed to assess the true incidence of primary non-response in children in a non clinical trial setting. The adult UC trials (ACT 1 and ACT 2) reported similar response rates among adult UC patients receiving infliximab as the CD trials. Infliximab is being used off label in children with UC and the official clinical trial for indication is currently underway. There are many differences in the patient population and outcome measures making a comparison across trials difficult and hard to interpret.

Inter-individual variability in therapeutic response may be best explained by genetic variability as it relates to disease pathogenesis and mechanism of action of this class of therapies. Other than NOD2 and IBD5, IBD susceptibility genes identified via genome wide linkage approach or Genome Wide Association Studies (GWAS) have not been evaluated as predictors of response to anti-TNFα therapies. NOD2 was not found to be associated with therapeutic response to infliximab in these limited studies. It is conceivable that disease susceptibility genes do not influence the ultimate response to therapeutic targets given the multifactorial influences on disease and the relatively unknown functionality of these susceptibility genes. However, the GWAS approach, which identifies portions of the genome that contain genetic variants associated with specific phenotypes, can also identity novel variants that contribute to therapeutic outcome i.e. discovery of genetic loci that are responsible for the mechanism of altered drug response, such as to anti-TNFα. There may also be important non genetic factors that influence or modify primary response to anti-TNFα. Among the serologic immune responses, pANCA has been shown to be negatively associated with primary response in both CD and UC patients. PANCA is present in both CD and UC and defines a specific colitis phenotype suggesting a degree of overlap in the underlying pathway biology of these two disease subtypes. Other than duration of disease at initiation of therapy, there are likely important clinical and demographic variables that also influence therapeutic outcomes. To date, however, it remains unknown whether these are independent of genetic variability. Thus, there is a need in the art to study associations of known IBD susceptibility loci as well as novel loci identified by pharmacogenetic GWAS with anti-TNFα response in pediatric IBD patients, and to develop predictive models of anti-TNFα primary non-response using clinical phenotype, serologic and genetic variables.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A-1F depicts, in accordance with embodiments herein, associations of genetic variants with primary non-response to anti-TNFα therapy as the outcome in patients with IBD. Results of SNPs with significance >10⁻³. FIG. 1A depicts, in accordance with embodiments herein, associations of genetic variants with primary non-response to anti-TNFα therapy as the outcome in patients with IBD. Results of SNPs with significance >10⁻³. FIG. 1B depicts, in accordance with embodiments herein, associations of genetic variants with primary non-response to anti-TNFα therapy as the outcome in patients with IBD. Results of SNPs with significance >10⁻³. FIG. 1C depicts, in accordance with embodiments herein, associations of genetic variants with primary non-response to anti-TNFα therapy as the outcome in patients with IBD. Results of SNPs with significance >10⁻³. FIG. 1D depicts, in accordance with embodiments herein, associations of genetic variants with primary non-response to anti-TNFα therapy as the outcome in patients with IBD. Results of SNPs with significance >10⁻³. FIG. 1E depicts, in accordance with embodiments herein, associations of genetic variants with primary non-response to anti-TNFα therapy as the outcome in patients with IBD. Results of SNPs with significance >10⁻³. FIG. 1F depicts, in accordance with embodiments herein, associations of genetic variants with primary non-response to anti-TNFα therapy as the outcome in patients with IBD. Results of SNPs with significance >10⁻³.

FIG. 2A-2F depicts, in accordance with embodiments herein, associations of genetic variants with secondary loss of response to anti-TNFα therapy as the outcome in patients with IBD. FIG. 2A depicts, in accordance with embodiments herein, associations of genetic variants with secondary loss of response to anti-TNFα therapy as the outcome in patients with IBD. FIG. 2B depicts, in accordance with embodiments herein, associations of genetic variants with secondary loss of response to anti-TNFα therapy as the outcome in patients with IBD. FIG. 2C depicts, in accordance with embodiments herein, associations of genetic variants with secondary loss of response to anti-TNFα therapy as the outcome in patients with IBD. FIG. 2D depicts, in accordance with embodiments herein, associations of genetic variants with secondary loss of response to anti-TNFα therapy as the outcome in patients with IBD. FIG. 2E depicts, in accordance with embodiments herein, associations of genetic variants with secondary loss of response to anti-TNFα therapy as the outcome in patients with IBD. FIG. 2F depicts, in accordance with embodiments herein, associations of genetic variants with secondary loss of response to anti-TNFα therapy as the outcome in patients with IBD.

FIG. 3A-3F depicts, in accordance with embodiments herein, associations of genetic variants with failure for any reason to anti-TNFα therapy as the outcome in patients with IBD. FIG. 3A depicts in accordance with embodiments herein, associations of genetic variants with failure for any reason to anti-TNFα therapy as the outcome in patients with IBD. FIG. 3B depicts in accordance with embodiments herein, associations of genetic variants with failure for any reason to anti-TNFα therapy as the outcome in patients with IBD. FIG. 3C depicts in accordance with embodiments herein, associations of genetic variants with failure for any reason to anti-TNFα therapy as the outcome in patients with IBD. FIG. 3D depicts in accordance with embodiments herein, associations of genetic variants with failure for any reason to anti-TNFα therapy as the outcome in patients with IBD. FIG. 3E depicts in accordance with embodiments herein, associations of genetic variants with failure for any reason to anti-TNFα therapy as the outcome in patients with IBD. FIG. 3F depicts in accordance with embodiments herein, associations of genetic variants with failure for any reason to anti-TNFα therapy as the outcome in patients with IBD.

FIG. 4 depicts, in accordance with embodiments herein, genotype associations of known IBD susceptibility loci with primary non-response. The dominant rare allele model (i.e. presence of the rare allele) was assumed for the statistical analyses. The relative risk (RR) and frequency of non-response are shown for the genotypes for each locus that met p value significance in univariate analysis.

FIG. 5 depicts, in accordance with embodiments herein, relative risk (RR) of non-response based on number of risk factors derived from Model V (the most general model). Risk of non-response was compared between patients with 2 or less risk factors as compared to 3-4 markers and then as compared to at least 5 of the 6 risk factors for non-response.

### SUMMARY OF THE INVENTION

Various embodiments include a method of determining a high risk relative to a normal subject of non-responsiveness to treatment with an anti tumor necrosis actor alpha (TNFα) therapy in an individual, comprising obtaining a sample from the individual, assaying the sample for the presence or absence of one or more genetic and/or serological risk factors, and determining the high risk relative to a normal subject of non-responsiveness to the anti TNFα therapy based on the presence of one or more risk factors carried by the individual. In another embodiment, the presence of each genetic and/or serological risk factor has an additive effect on increasing the risk of non-responsiveness in the individual. In another embodiment, the individual is diagnosed with inflammatory bowel disease (IBD). In another embodiment, the individual is diagnosed with ulcerative colitis (UC). In another embodiment, the individual is a child. In another embodiment, the one or more genetic risk factors comprise genetic variants at the loci of tachykinin receptor 1 (TACR1), family with sequence similarity 19 member A4 (FAM19A4), phosphatase and actin regulator 3 (PHACTR3) and/or bromodomain and WD repeat domain containing 1 (BRWD1). In another embodiment, the one or more genetic risk factors comprise SEQ. ID. NO.: 1, SEQ. ID. NO.: 2, SEQ. ID. NO.: 3, SEQ. ID NO.: 4, SEQ. ID. NO.: 5 and/or SEQ. ID. NO.: 6. In another embodiment, the one or more genetic risk factors comprise SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, and/or SEQ. ID. NO.: 16. In another embodiment, the one or more genetic risk factors comprise SEQ. ID. NO.: 17, SEQ. ID. NO.: 8, SEQ. ID. NO.: 19, and/or SEQ. ID. NO.: 6. In another embodiment, the one or more genetic risk factors comprise genetic variants at the loci of ATG16, Orf13, inducible T-cell co-stimulator ligand (ICOSLG) and/or major histocompatibility complex class II DQ alpha 1 (HLADQA1). In another embodiment, one of the one or more serological risk factors comprise perinuclear anti-neutrophil cytoplasmic antibody (pANCA). In another embodiment, the anti TNFα therapy comprises infliximab. In another embodiment, the anti TNFα therapy comprises cyclosporin.

Other embodiments include a method of determining a significant likelihood of responsiveness to treatment with anti tumor necrosis factor alpha (TNF-α) therapy in an individual, comprising obtaining a sample from the individual, assaying the sample for the presence of one or more serological markers associated with responsiveness to anti TNFα therapy, and determining a significant likelihood of responsiveness based on the presence of one or more serological markers associated with responsiveness to anti TNFα therapy. In another embodiment, the individual is diagnosed with inflammatory bowel disease (IBD). In another embodiment, the individual is diagnosed with ulcerative colitis (UC). In another embodiment, the individual is a child. In another embodiment, one of the one or more serological markers comprises anti-Saccharomyces cerevisiae antibodies (ASCA).

Other embodiments include a method of predicting a high risk relative to a normal subject of non-responsiveness to anti tumor necrosis factor alpha (TNF-α) therapy in an individual with inflammatory bowel disease (IBD), comprising determining the presence or absence of one or more nonresponsive genetic risk variants, determining the presence or absence of positive expression of perinuclear anti-neutrophil cytoplasmic antibody (pANCA), determining the presence or absence of an ulcerative colitis phenotype, and predicting a high risk relative to a normal subject of non responsiveness to anti TNF-α therapy based on the presence of one or more responsive risk variants, the presence of positive expression of pANCA, and/or the presence of the ulcerative colitis phenotype. In another embodiment, one of the one or more nonresponsive genetic risk variants comprise variants at the genetic loci of tachykinin receptor 1 (TACR1), family with sequence similarity 19 member A4 (FAM19A4), phosphatase and actin regulator 3 (PHACTR3) and/or bromodomain and WD repeat domain containing 1 (BRWD1). In another embodiment, the high risk relative to a normal subject of non-responsiveness comprises a range of 7 to 10 fold increase in risk of non-responsiveness to treatment with anti TNFα therapy.

Various embodiments include a method of diagnosing an inflammatory bowel disease (IBD) subtype in an individual, comprising obtaining a sample from the individual, assaying the sample for the presence or absence of one or more genetic and/or serological risk factors of nonresponsiveness to anti TNFα therapy, and diagnosing the IBD subtype based upon the presence of one or more genetic and/or serological risk factors of nonresponsiveness to anti TNFα therapy. In another embodiment, the individual is a child. In another embodiment, the one or more genetic risk factors comprise genetic variants at the loci of tachykinin receptor 1 (TACR1), family with sequence similarity 19 member A4 (FAM19A4), phosphatase and actin regulator 3 (PHACTR3) and/or bromodomain and WD repeat domain containing 1 (BRWD1). In another embodiment, the one or more genetic risk factors comprise SEQ. ID. NO.: 1, SEQ. ID. NO.: 2, SEQ. ID. NO.: 3, SEQ. ID NO.: 4, SEQ. ID. NO.: 5 and/or SEQ. ID. NO.: 6. In another embodiment, the one or more genetic risk factors comprise SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, and/or SEQ. ID. NO.: 16. In another embodiment, the one or more genetic risk factors comprise SEQ. ID. NO.: 17, SEQ. ID. NO.: 8, SEQ. ID. NO.: 19, and/or SEQ. ID. NO.: 6. In another embodiment, one of the one or more serological risk factors comprise perinuclear anti-neutrophil cytoplasmic antibody (pANCA).

Other embodiments include a method of treating an individual, comprising diagnosing the individual as susceptible to non-responsiveness to anti tumor necrosis factor alpha (TNF-α) therapy, and treating the individual. In another embodiment, treating the individual comprises administering a therapeutically effective dosage of natalizumab. In another embodiment, the individual has inflammatory bowel disease (IBD).

Other features and advantages of the invention will become apparent from the following detailed description, taken in conjunction with the accompanying drawings, which illustrate, by way of example, various embodiments of the invention.

### DESCRIPTION OF INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, NY 1994); March, Advanced Organic Chemistry Reactions, Mechanisms and Structure 4th ed., J. Wiley & Sons (New York, NY 1992); and Sambrook and Russel, Molecular Cloning: A Laboratory Manual 3rd ed., Cold Spring Harbor Laboratory Press (Cold Spring Harbor, NY 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

One skilled in the art will recognize many methods and materials similar or equivalent to those described herein, which could be used in the practice of the present invention. Indeed, the present invention is in no way limited to the methods and materials described.
“IBD” as used herein is an abbreviation of inflammatory bowel disease.
“CD” as used herein is an abbreviation of Crohn's Disease.
“UC” as used herein is an abbreviation of ulcerative colitis.
“GWA” as used herein is an abbreviation of genome wide associations.
“IFX” as used herein is an abbreviation of infliximab.
“TNFα” as used herein is an abbreviation of tumor necrosis factor alpha.
“SNP” as used herein is an abbreviation of single-nucleotide polymorphism
“ATI” as used herein is an abbreviation of anti infliximab antibodies.
“CDAI” as used herein is an abbreviation of Crohn's Disease activity index.
“PCDAI” as used herein is an abbreviation of pediatric Crohn's Disease activity index.
“pANCA” as used herein is an abbreviation of perinuclear anti-neutrophil cytoplasmic antibodies.
“ASCA” as used herein is an abbreviation of Anti-Saccharomyces cerevisiae antibodies.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As used herein, the term “normal subject” means an individual who has an average likelihood of successful treatment.

As used herein, the term “positive likelihood ratio of non-response” means the value that when multiplied by the odds of non-responsiveness in a normal subject yields the total odds of non-responsiveness in an individual.

As readily apparent to one of skill in the art, any number of examples may be used for various genetic loci and variants described herein and the invention is in no way limited to specific examples of sequences used and described herein. For example, SNPs rs2241880, rs2188962, rs3764147, rs762421, rs9271568, rs2836878, described herein as SEQ. ID. NO.: 1, SEQ. ID. NO.: 2, SEQ. ID. NO.: 3, SEQ. ID NO.: 4, SEQ. ID. NO.: 5, and SEQ. ID. NO.: 6, respectively, are not limited to the specific sequences described and various additional genetic sequences may also be used while still containing the relevant allele. Similarly, as apparent to one of skill in the art, various examples of sequences may be used to represent SNPs rs13079040, rs4855535, rs17048128, rs17048129, rs17039556, rs12640159, rs880330, rs2057917, rs2983478, rs4776127, rs975664, rs6100556, and rs2836878, and thus the aforementioned genetic variants are not specifically limited to the sequences described herein as SEQ. ID. NO.: 7, SEQ. ID. NO.: 8, SEQ. ID. NO.: 9, SEQ. ID. NO.: 10, SEQ. ID. NO.: 11, SEQ. ID. NO.: 12, SEQ. ID. NO.: 13, SEQ. ID. NO.: 14, SEQ. ID. NO.: 15, and SEQ. ID. NO.: 16, SEQ. ID. NO.: 17, SEQ. ID. NO. 18, and SEQ. ID. NO.: 19, respectively.

As further described herein, inter-individual variation in response to anti-TNFα therapy may be explained by genetic variability in disease pathogenesis or mechanism of action. Recent genome wide association studies (GWAS) in IBD have increased understanding of the genetic susceptibility to IBD.

As described herein, the inventors have developed various predictive models, including a predicted model of primary response by testing associations of known IBD susceptibility loci and novel “pharmacogenetic” GWAS identified loci with primary non-response to anti-TNFα in pediatric IBD patients. Primary non response was defined using the HBI for CD and partial Mayo score for UC. Genotyping was performed using the Illumina Infinium platform. Chi square analysis tested associations of phenotype and genotype with primary non-response. Genetic associations were identified by testing known IBD susceptibility loci and by performing a GWAS for primary non-response. Step-wise multiple logistic regression was performed to build predictive models.

As further described herein, non-response occurred in 22 of 94 subjects. Six known susceptibility loci were associated with primary non-response (p<0.05). The 21q22.2/BRWD/loci remained significant in the predictive model. The most predictive model included 3 novel “pharmacogenetic” GWAS loci, previously identified BRWD1, PANCA and a UC diagnosis (R²=0.82 and AUC=0.98%). The relative risk of non-response increased 15 fold when number of risk factors increased from 0-2 to ≥3.

As further disclosed herein, the inventors have described the combination of phenotype and genotype as most predictive of primary non response to anti-TNFα in pediatric IBD. Defining predictors of response to anti-TNFα allows the identification of patients who will not benefit from this class of therapy.

In one embodiment, the present invention provides a method of evaluating the prognosis of anti-TNFα therapy in an individual undergoing inflammatory bowel disease treatment by determining the presence or absence of a non-response genetic variant and/or non-response serological marker, where the presence of the non-response genetic variant and/or non-response serological marker is indicative of inflammatory bowel disease that is non-responsive to anti-TNFα therapy. In another embodiment, the non-response genetic variant and/or non response serological marker is described in Tables 1, 2 and/or 3 (A)-(D). In another embodiment, the non-response genetic variant is at the genetic loci of ATG16, Orf13, ICOSLG, HLADQA1 and/or BRWD1. In another embodiment, the non-response serological marker is pANCA. In another embodiment, the anti-TNFα therapy includes the use of Infliximab. In another embodiment, the individual is a child.

In one embodiment, the present invention provides a method of evaluating the prognosis of anti-TNFα therapy in an individual undergoing inflammatory bowel disease treatment by determining the presence or absence of a non-response genetic variant and/or a response serological marker, where the absence of the non-response genetic variant and/or the presence of the response serological marker is indicative of inflammatory bowel disease responsive to anti-TNFα therapy. In another embodiment, the non-response genetic variant and/or response serological marker is described in Tables 1, 2 and/or 3 (A)-(D). In another embodiment, the non-response genetic variant is at the genetic loci of ATG16, Orf13, ICOSLG, HLADQA1 and/or BRWD1. In another embodiment, the response serological marker is ASCA. In another embodiment, the anti-TNFα therapy includes the use of Infliximab. In another embodiment, the individual is a child.

In one embodiment, the present invention provides a method of diagnosing an inflammatory bowel subtype in an individual by determining the presence or absence of a non-response genetic variant and/or non-response serological marker, where the presence of the non-response genetic variant and/or non-response serological marker is indicative of the inflammatory bowel disease subtype in the individual. In another embodiment, the individual is a child.

In one embodiment, the present invention provides a method of treating inflammatory bowel disease in an individual by determining the presence of a non-response genetic variant and/or non-response serological marker, and treating the individual. In another embodiment, the individual is a child. In another embodiment, the treatment includes the use of anti-TNFα therapy.

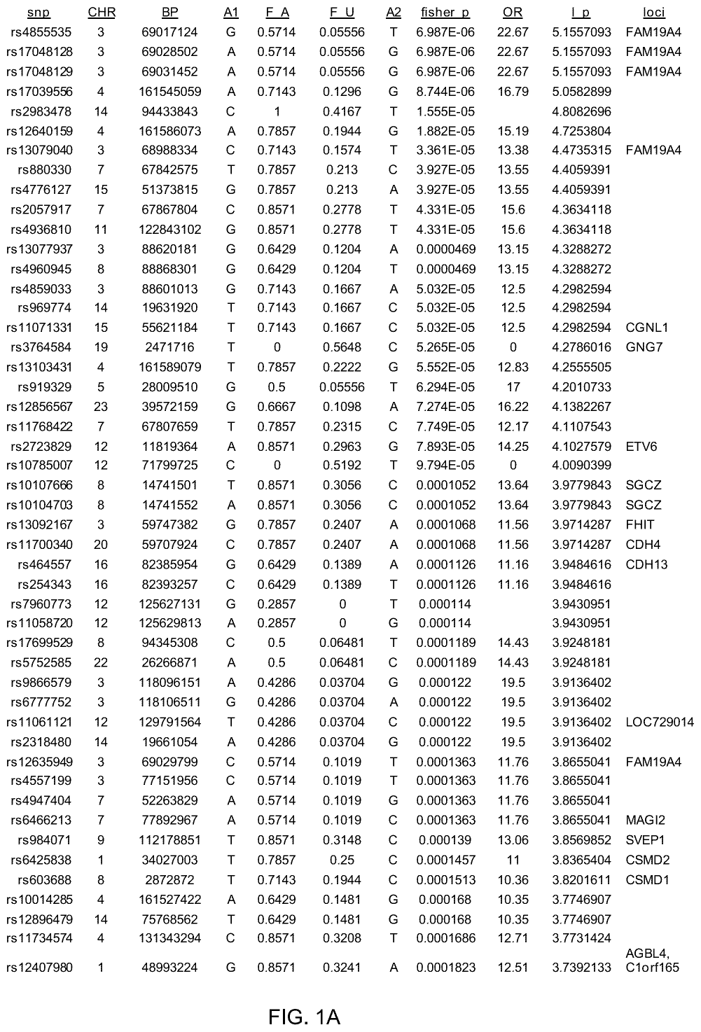

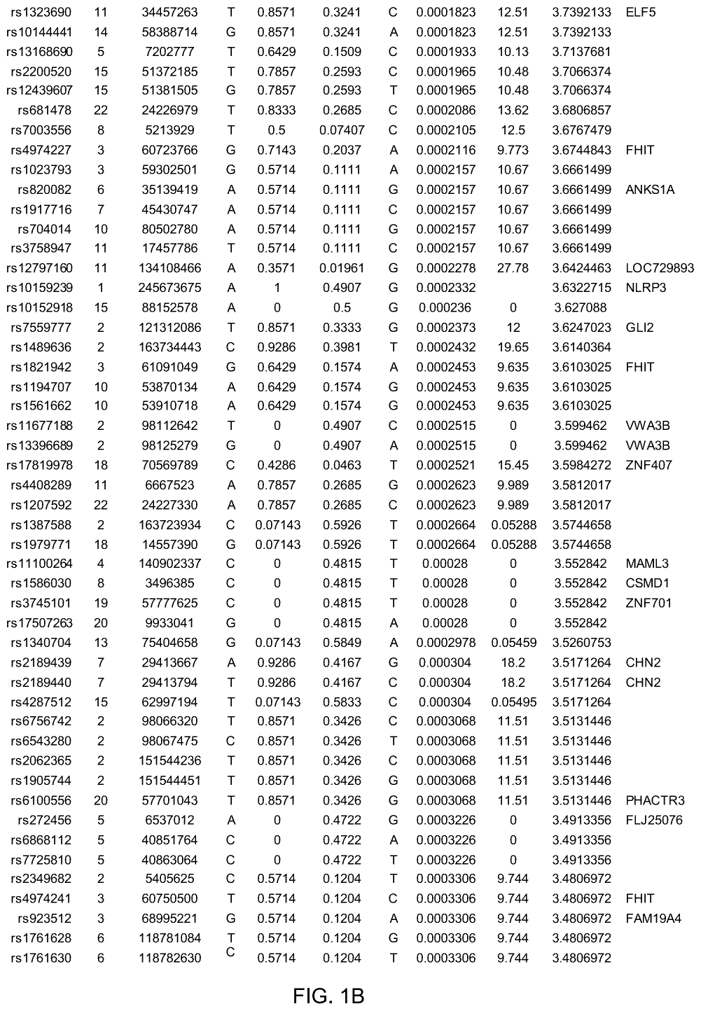

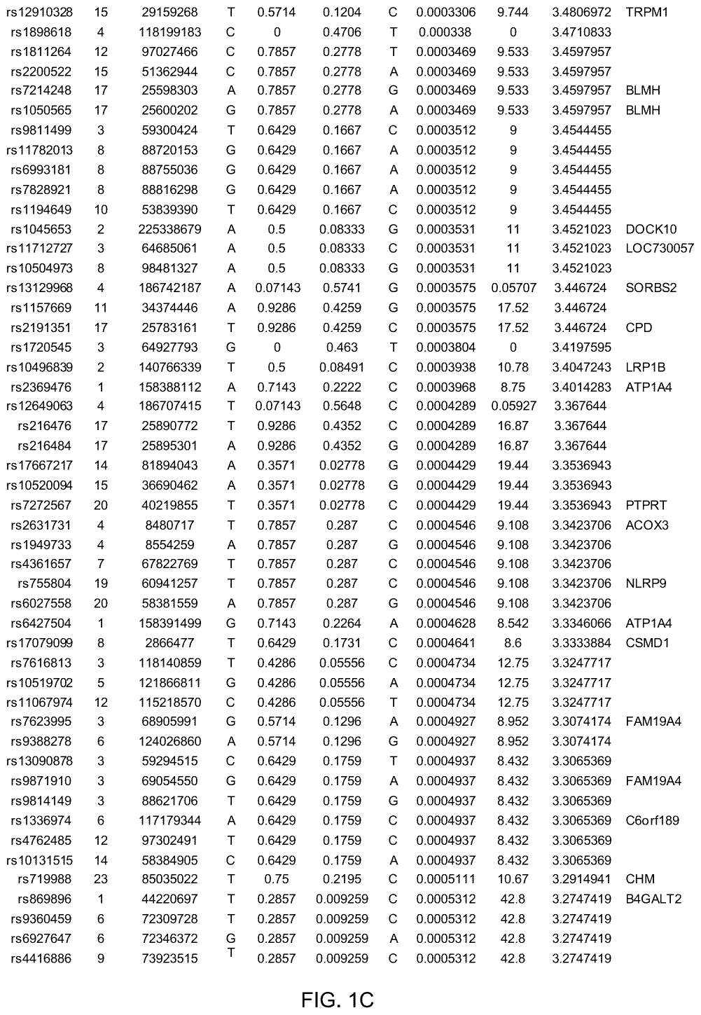

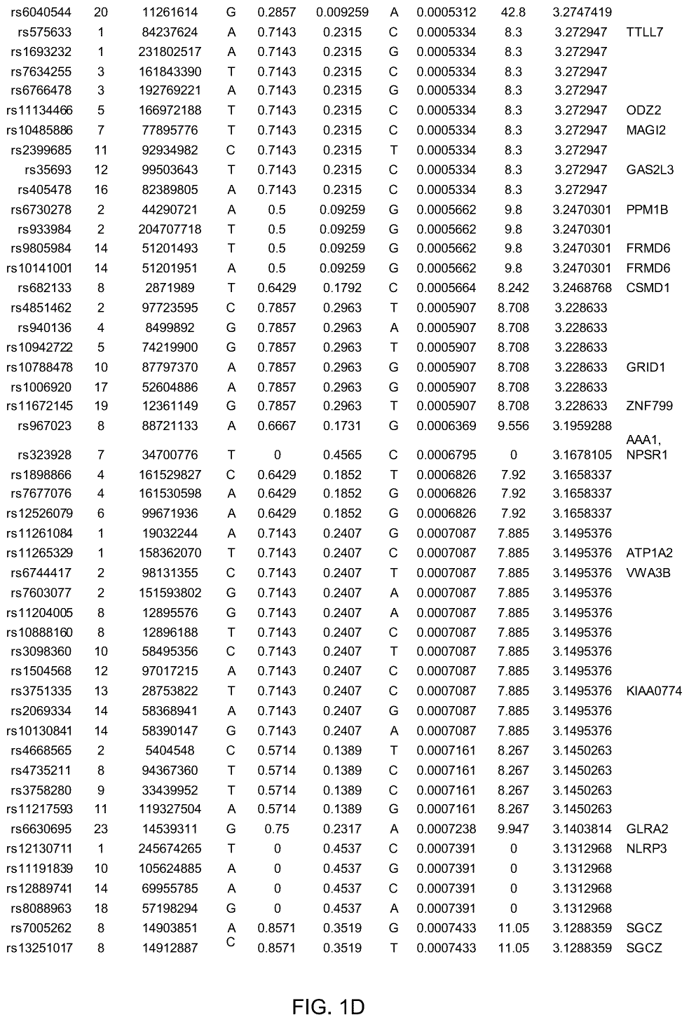

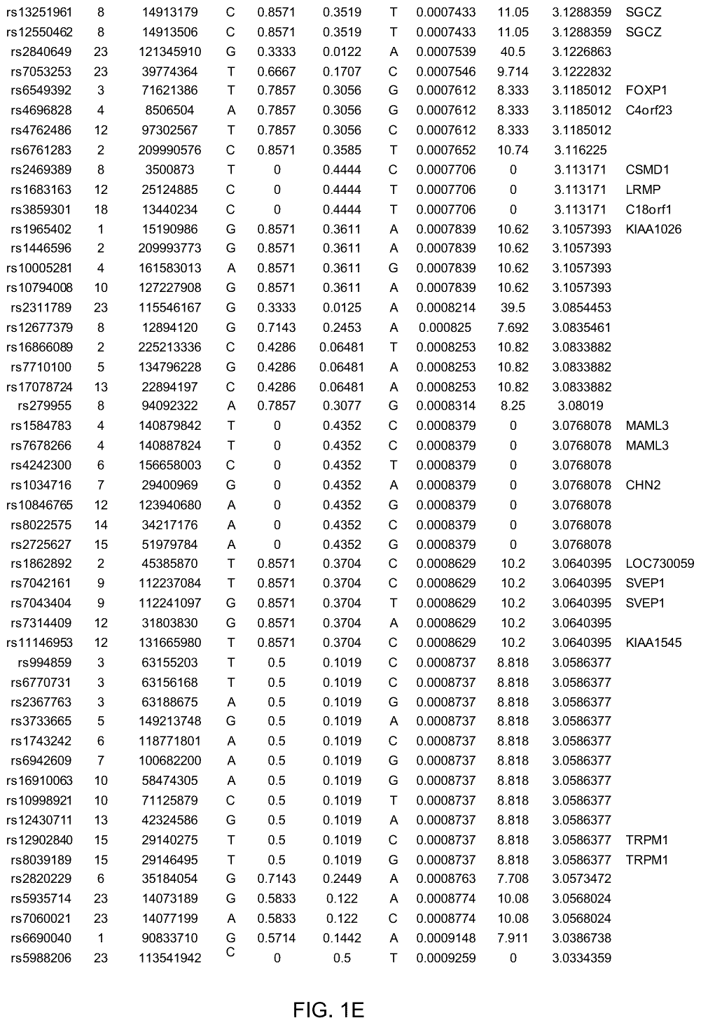

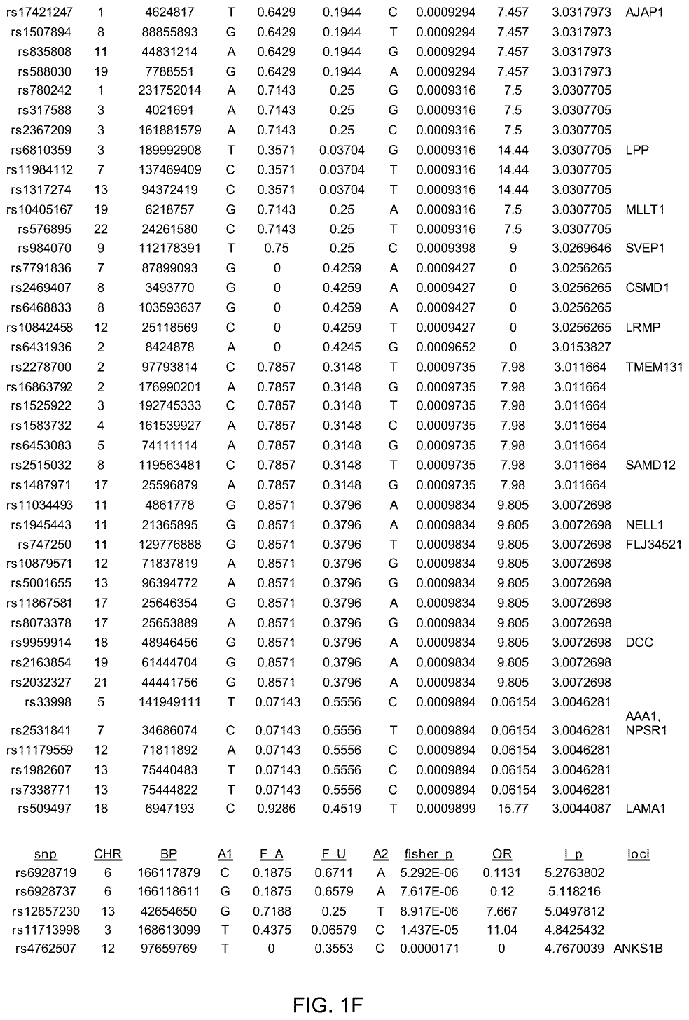

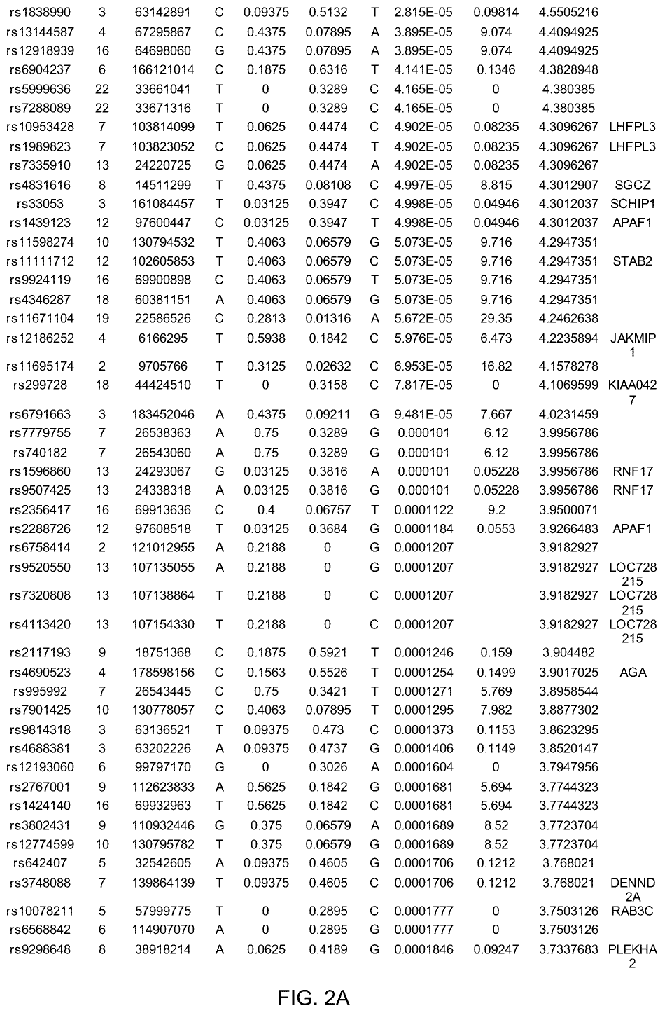

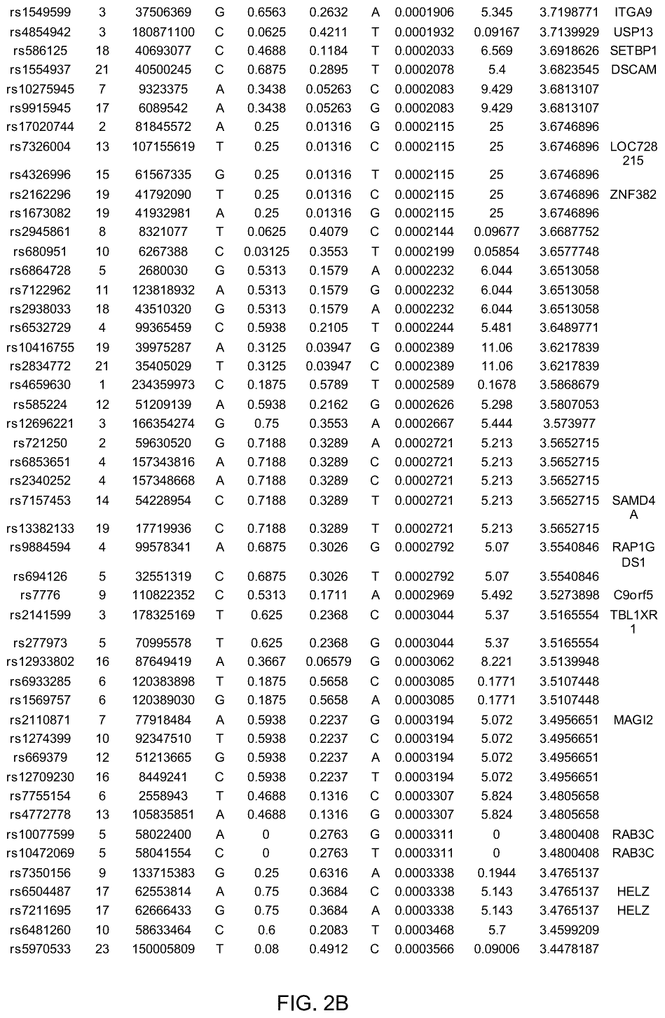

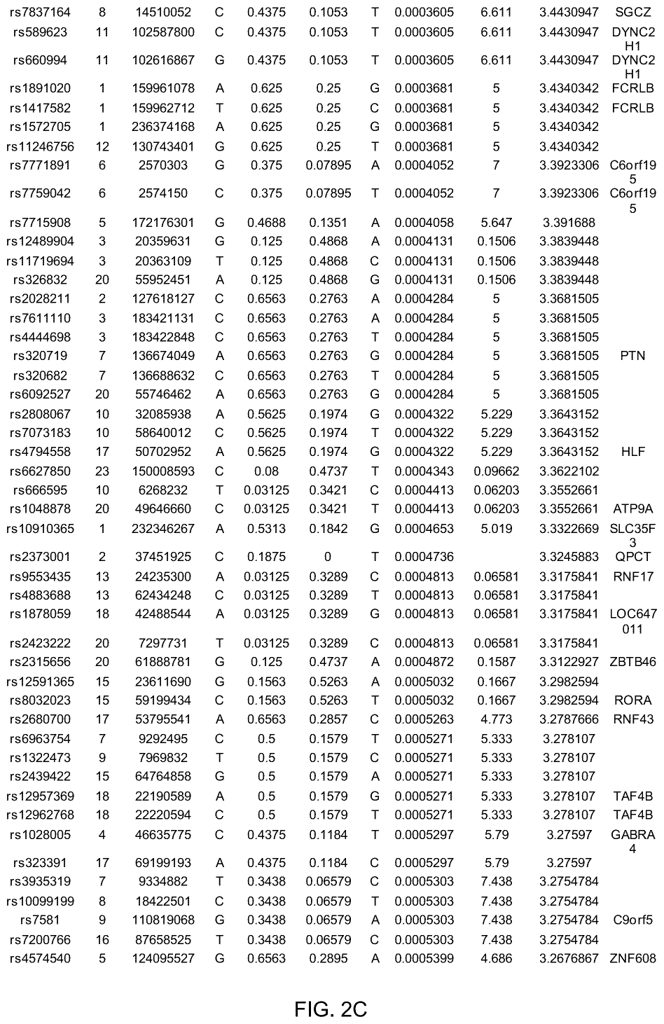

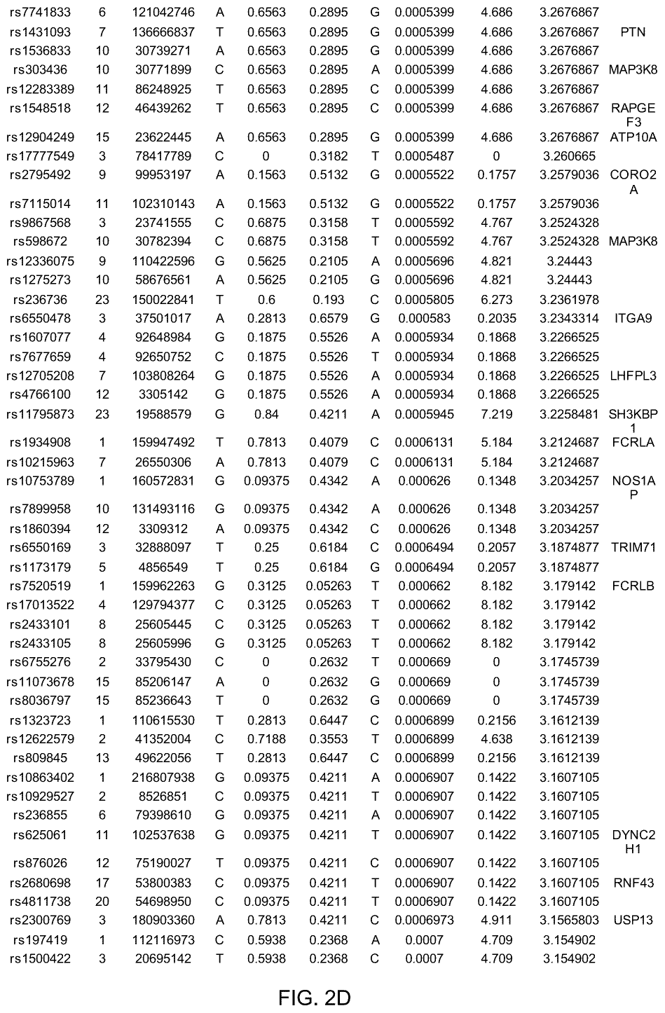

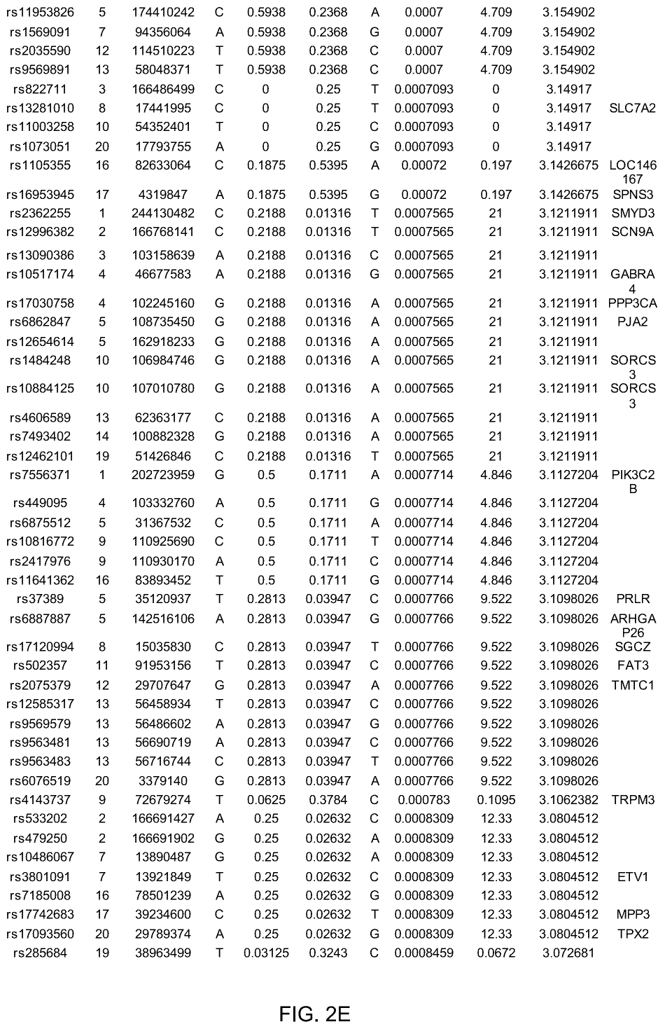

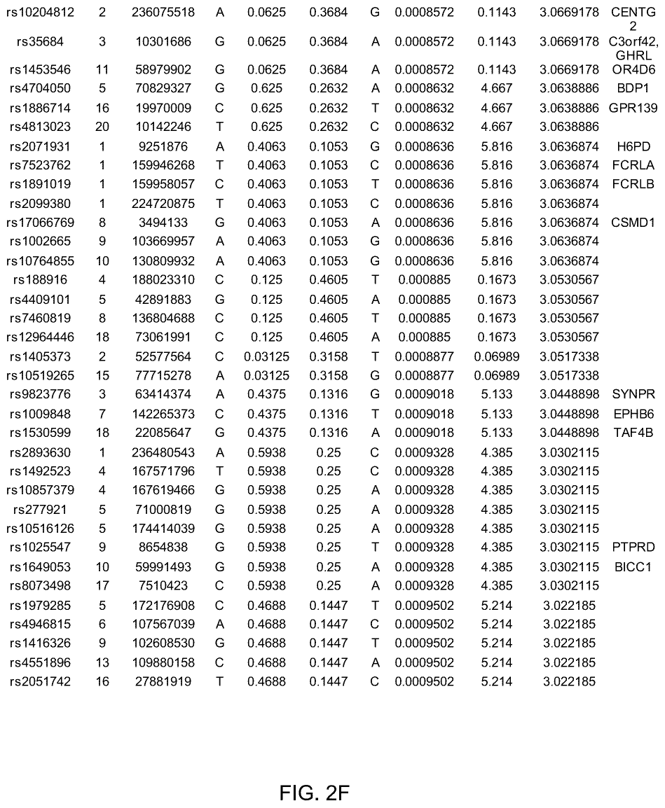

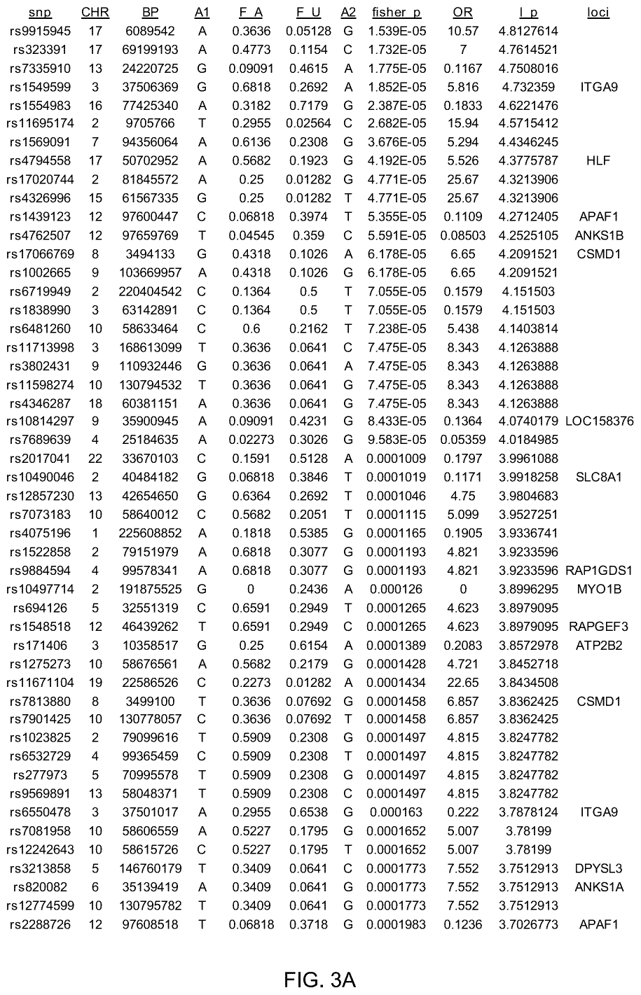

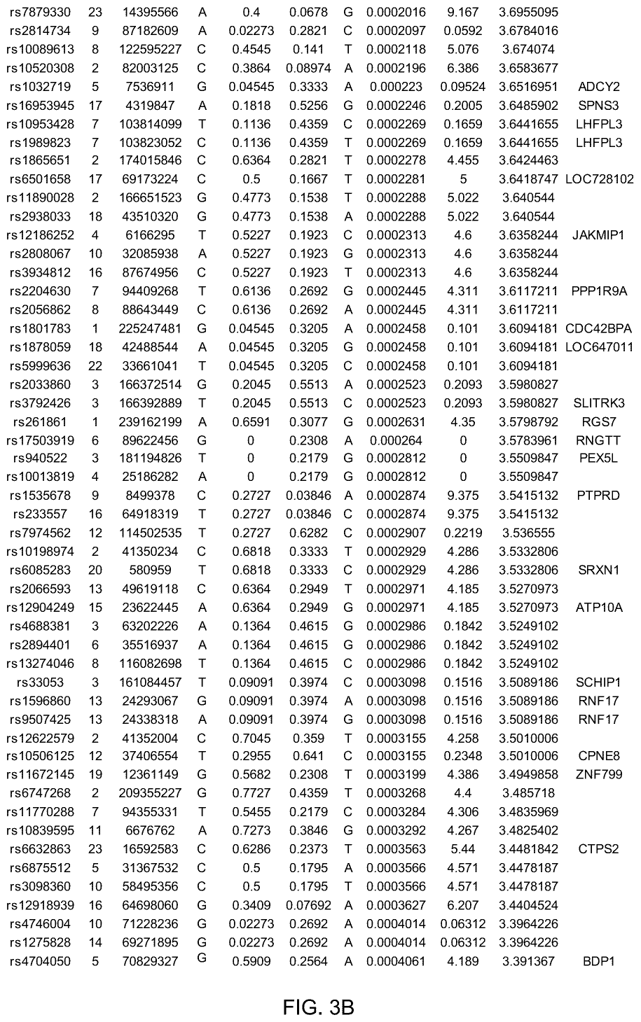

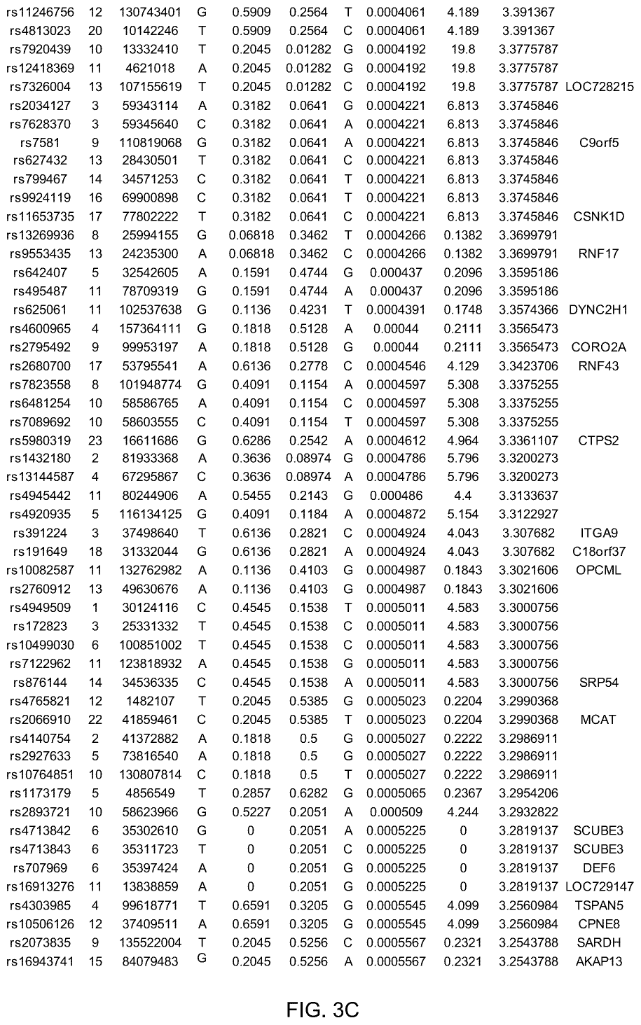

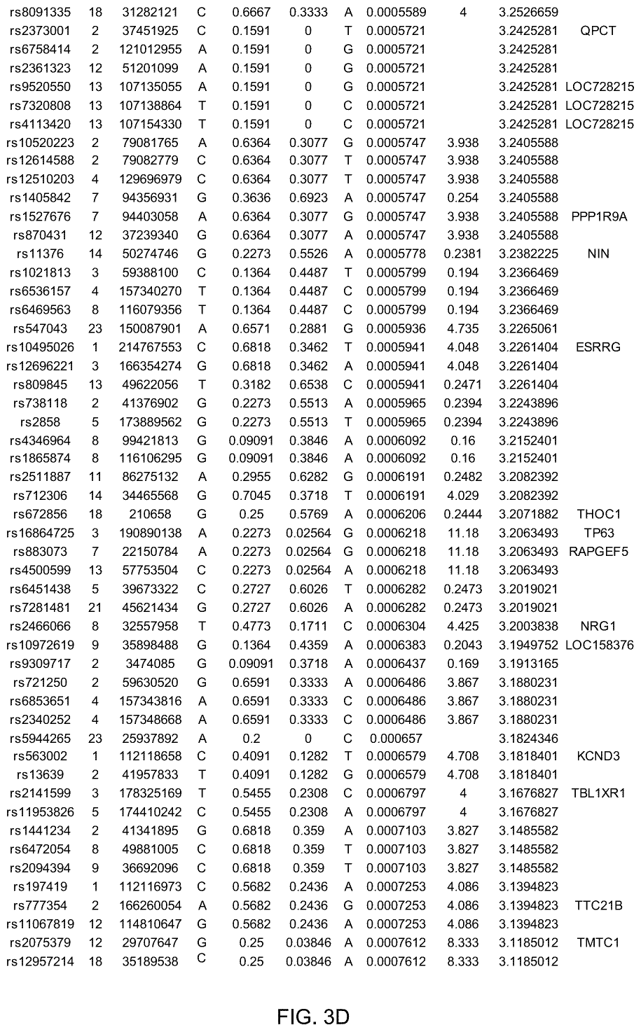

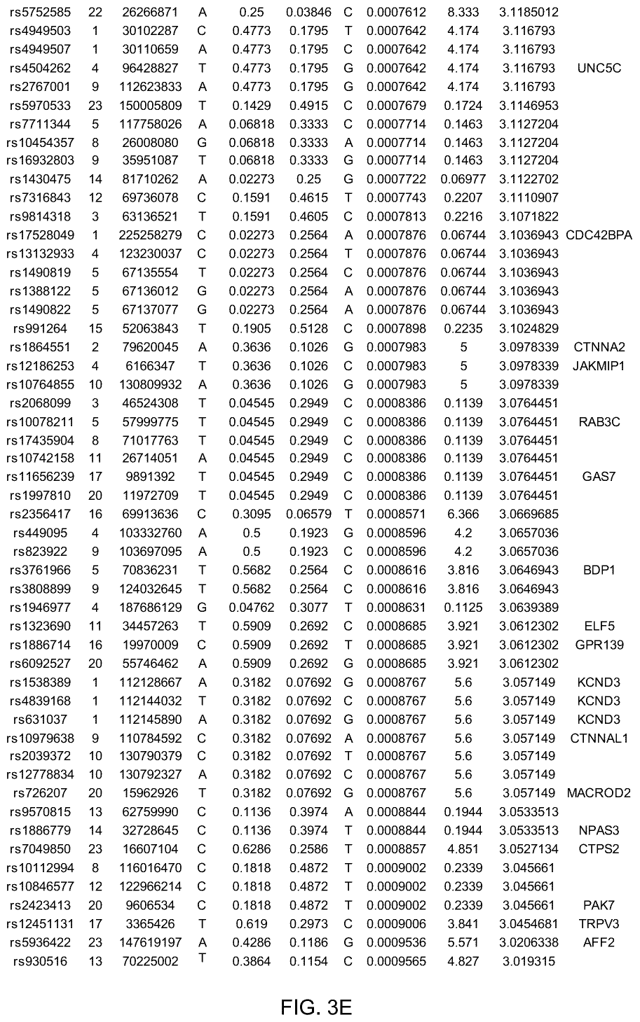

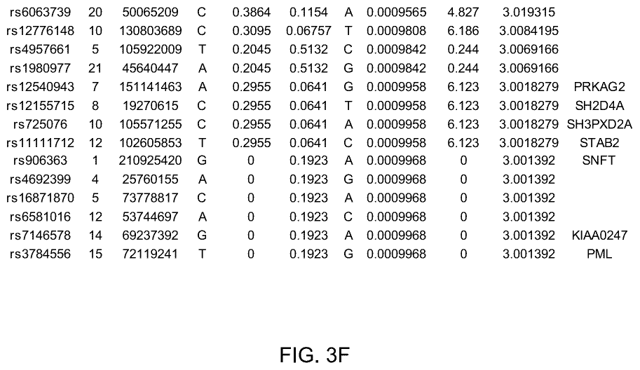

As disclosed herein, the inventors conducted association studies of anti-TNFα responsiveness against the whole genome. Three (3) outcomes were evaluated (primary non-response, loss of response, and failure for any reason), with the analysis of such outcomes described in Table 4 and FIGS. 1-3 herein, including novel findings in the FAM19 genetic locus.

In one embodiment, the present invention provides a method of evaluating the prognosis of anti-TNFα therapy in an individual undergoing inflammatory bowel disease treatment by determining the presence or absence of a non-response genetic variant, where the presence of the non-response genetic variant is indicative of inflammatory bowel disease that is non-responsive to anti-TNFα therapy. In another embodiment, the non-response genetic variant is described in Table 4 and/or FIGS. 1-3 herein. In another embodiment, the anti-TNFα therapy includes the use of Infliximab. In another embodiment, the individual is a child.

In one embodiment, the present invention provides a method of evaluating the prognosis of anti-TNFα therapy in an individual undergoing inflammatory bowel disease treatment by determining the absence of a non-response genetic variant, where the absence of the non-response genetic variant is indicative of inflammatory bowel disease responsive to anti-TNFα therapy. In another embodiment, the non-response genetic variant is described in Table 4 and/or FIGS. 1-3. In another embodiment, the response genetic variant is at the FAM19A4 genetic locus. In another embodiment, the anti-TNFα therapy includes the use of Infliximab. In another embodiment, the individual is a child.

As disclosed herein, the inventors tested associations of genetic loci with anti-TNFα response in pediatric IBD patients by pursing a variety of strategies. The result was the development of various predictive models of anti-TNFα response using phenotype, serologic and genetic variables.

In one embodiment, the present invention provides a method of evaluating the prognosis and/or predicting responsiveness of anti-TNFα therapy in an individual undergoing inflammatory bowel disease treatment by determining the presence or absence of a non-response genetic variant, where the presence of the non-response genetic variant is indicative of inflammatory bowel disease that is non-responsive to anti-TNFα therapy. In another embodiment, the non-response genetic variant is a known IBD susceptibility locus. In another embodiment, the non-response genetic variant is described in Tables 6-10 herein. In another embodiment, the anti-TNFα therapy includes the use of Infliximab. In another embodiment, the individual is a child.

In another embodiment, the present invention provides a method of evaluating the prognosis and/or predicting responsiveness of anti-TNFα therapy in an individual undergoing inflammatory bowel disease treatment by determining the presence or absence of one or more risk factors, where the presence of each risk factor has an additive effect for an increased risk of non-responsiveness to anti-TNFα therapy. In another embodiment, one of the risk factors is a non-response genetic variant. In another embodiment, the non-response genetic variant is described in Tables 6-10 herein. In another embodiment, one of the risk factors is positive expression of a serological marker. In another embodiment, the serological marker is pANCA. In another embodiment, one of the risk factors is the diagnosis of the individual, where a diagnosis of ulcerative colitis predicts non-responsiveness to anti-TNFα therapy. In another embodiment, the anti-TNFα therapy includes the use of Infliximab. In another embodiment, the individual is a child.

In one embodiment, the present invention provides a method of treating inflammatory bowel disease in an individual by determining the presence of one or more risk factors and treating the individual. In another embodiment, one of the risk factors is a non-response genetic variant. In another embodiment, the non-response genetic variant is described in Tables 6-10 herein. In another embodiment, one of the risk factors is positive expression of a serological marker. In another embodiment, the serological marker is pANCA. In another embodiment, one of the risk factors is the diagnosis of the individual, where a diagnosis of ulcerative colitis predicts non-responsiveness to anti-TNFα therapy. In another embodiment, the individual is a child. In another embodiment, the treatment includes the administration of a therapeutically effective amount of anti-TNFα therapy to the individual.

In one embodiment, the present invention provides a method of diagnosing an inflammatory bowel subtype in an individual by determining the presence or absence of one or more risk factors, where the presence of one or more risk factors is indicative of the inflammatory bowel disease subtype in the individual. In another embodiment, one of the risk factors is a non-response genetic variant. In another embodiment, the non-response genetic variant is described in Tables 6-10 herein. In another embodiment, one of the risk factors is positive expression of a serological marker. In another embodiment, the serological marker is pANCA. In another embodiment, one of the risk factors is the diagnosis of the individual, where a diagnosis of ulcerative colitis predicts non-responsiveness to anti-TNFα therapy. In another embodiment, the individual is a child.

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

Similarly, there are many techniques readily available in the field for detecting the presence or absence of serotypes, antibodies, polypeptides or other biomarkers, including protein microarrays. For example, some of the detection paradigms that can be employed to this end include optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Similarly, there are any number of techniques that may be employed to isolate and/or fractionate biomarkers. For example, a biomarker may be captured using biospecific capture reagents, such as antibodies, aptamers or antibodies that recognize the biomarker and modified forms of it. This method could also result in the capture of protein interactors that are bound to the proteins or that are otherwise recognized by antibodies and that, themselves, can be biomarkers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer and hybrids of these.

Alternatively, for example, the presence of biomarkers such as polypeptides maybe detected using traditional immunoassay techniques. Immunoassay requires biospecific capture reagents, such as antibodies, to capture the analytes. The assay may also be designed to specifically distinguish protein and modified forms of protein, which can be done by employing a sandwich assay in which one antibody captures more than one form and second, distinctly labeled antibodies, specifically bind, and provide distinct detection of, the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, biomarkers may also be fractionated to isolate them from other components in a solution or of blood that may interfere with detection. Fractionation may include platelet isolation from other blood components, sub-cellular fractionation of platelet components and/or fractionation of the desired biomarkers from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Generally

Genetics, immune responses and environmental factors for disease susceptibility and development, as well as their interactions, are important determinants of inflammatory bowel disease phenotype and disease progression. These factors may also interact in such a way that influences the outcome of therapies used to treat these heterogeneous phenotypes. Recent genomic discoveries from Genome Wide Association (GWA) studies in both Crohn's disease (CD) and ulcerative colitis (UC) have increased understanding of the genetic susceptibility to IBD. This novel genetic information provides important insight regarding the various mechanisms of inflammation involved in disease pathogenesis. Targeting these various pathways with effective therapies is the key to the successful management of the IBD patient. When introduced, the monoclonal antibodies targeting tumor necrosis factor alpha (TNFα) represented the largest advance in decades made in the realm of IBD therapeutics. However there is clear inter-individual variability in both efficacy and safety outcomes to this class of therapy which has yet to be explained. The inventors have demonstrated that therapeutic outcomes to anti-TNFα in children and young adults with IBD are associated with inter-individual genetic variability, and determined that genetic loci identified by Genome Wide Association (GWA) studies alone or in combination with clinical and/or immune markers are associated with and predictive of therapeutic responsiveness to anti-TNFα therapy in pediatric IBD patients.

### Example 2

### Significance of Defining Predictors of Response to Anti-TNFα

Defining predictors of response to anti-TNFα will allow clinicians to choose the appropriate therapy for the appropriate IBD patient with the goal of maximizing efficacy and minimizing toxicity. Research described herein will allow the individualization of therapy based on who will or perhaps more importantly will not respond to different classes of therapeutic interventions currently available to IBD patients. The development of lymphoma, particularly a rare almost uniformly fatal sub-type of hepatosplenic T cell lymphoma in individuals receiving infliximab along with immunomodulators have resulted in clinicians wanting to carefully select those patients who are appropriate candidates for these therapies. The novel pharmacogenetic information described herein can not only improve the management of patients in the clinic with an existing anti-TNFα agent but also ultimately change the way large scale clinical trials are conducted, such that only patients with a higher probability of response to specific therapies will be enrolled to negate exposure to ineffective therapies and protect patients from treatment related serious and potentially fatal adverse events. The data described herein will aid in the translation of significant genetic findings into the clinical setting for IBD patients and for other patients receiving anti-TNFα for other immune mediated disorders.

### Example 3

### Pharmacogenetic GWAS and Primary Non-Response

The inventors tested the association of the most significant CD susceptibility loci previously identified with infliximab responsiveness in pediatric IBD patients receiving infliximab from which there was complete clinical follow up. For these preliminary analyses, two (2) outcomes were evaluated:
1) primary non-response: patient did not respond to the induction regimen as defined by patient did not receive a clinical benefit from the first 3 infusions of infliximab and did not receive any further treatment doses. All significant associations are shown in Table 1 below. Remainder of analyses are detailed in Table 3.
2) secondary loss of response: patient responded to the induction regimen and despite dose escalation and/or frequency intensification of infliximab the drug was discontinued as of last follow up. Time to loss of response was also analyzed and data are shown in Table 3.

### Example 4

### Table 1—Significant Associations Between GWAS Loci and Primary Non-Response

TABLE 1 (A)

| | | Primary Non | | | | | |
|---|---|---|---|---|---|---|---|
| | | Response (YES = | Genotype | Genotype | P | | Gene of |
| SNP | Chromosome | 1, NO = 0) | 12/22 | 11 | value | OR* | interest |
| rs2241880 | 2q37 | 0 | 30(58.8%) | 21(41.2%) | 0.04 | | ATG16 |
| | | 1 | 7(100.0%) | | | 10.6 | |
| rs2188962 | 5q23 | 0 | 45(83.3%) | 9(16.7%) | 0.03 | 6.7 | unknown |
| | | 1 | 3(42.9%) | 4(57.1%) | | | |
| rs3764147 | 13q14 | 0 | 31(57.4%) | 23(42.6%) | 0.004 | 20.1 | Orf13 |
| | | 1 | | 7100.0%) | | | |
| rs762421 | 21q22 | 0 | 34(63.0%) | 20(37.0%) | 0.03 | 10.2 | ICOSLG |
| | | 1 | 1(14.3%) | 6(85.7%) | | | |
| rs9271568 | 6p21.32 | 0 | 31(58.5%) | 22(41.5%) | 0.004 | 21.0 | HLADQA1 |
| | | 1 | | 7(100.0%) | | | |
| rs2836878 | 21q22.2 | 0 | 29(53.7%) | 25(46.3%) | 0.01 | 17.4 | BRWD1 |
| | | 1 | | 7(100.0%) | | | |

*If there is any zero cell, 0.5 is added to each cell count to calculate OR.

As described in Table 1 (A), there were 6 SNPs that were found to be significantly associated with primary non-response. Interestingly there was no overlap with any SNP found to be associated with secondary loss of response and time to loss of response. This shows that there may be different genetic predictors and biological explanations for the 2 therapeutic response outcomes.

In conjunction with the various genotypes and SNPs listed in Table 1 (A) above, the alleles are listed in Table 1 (B) below:

TABLE 1(B)

| SNP | Alleles Corresponding to Genotype |
|---|---|
| rs2241880 | 11 = CC, 12 = CT, 22 = TT |
| rs2188962 | 11 = CC, 12 = CT, 22 = TT |
| rs3764147 | 11 = AA, 12 = AG, 22 = GG |
| rs762421 | 11 = AA, 12 = AG, 22 = GG |
| rs9271568 | 11 = GG, 12 = GA, 22 = AA |
| rs2836878 | 11 = GG, 12 = GA, 22 = AA |

### Example 5

### Serological Immune Responses and Therapeutic Response

The associations between ASCA, pANCA, OmpC, I2 and CBIr-1 antibodies and therapeutic outcome was analyzed. Only significant associations with primary non-response are illustrated in Table 2. pANCA positivity was associated with primary non-response and ASCA positivity was protective against primary non-response. There was no association found anti-OmpC, anti-12 and anti-CBir-1 for primary non-response and none of the serologies were associated with loss of response.

### Example 6

### Table 2—Significant Associations Between Serological Immune Responses and Therapeutic Outcome

TABLE 2

| | Positive = 1, | Primary non response | | | | |
|---|---|---|---|---|---|---|
| Immune Responses | Negative = 0 | yes | no | P | OR* | 95% CI |
| pANCA | 0 | 3(30.0%) | 44(69.8%) | | | |
| | 1 | 6(85.7%) | 12(25.5%) | 0.002 | 17.5 | 1.9-160.6 |
| ASCA IgA ancifor IgG | 0 | 1(14.3%) | 35(74.5%) | | | |
| | 1 | | 30(47.6%) | 0.004 | 0.05 | |

*If there is any zero cell, 0.5 is added to each cell count to calculate OR.

### Example 7

### Table 3 (A)-(D)—Details of SNPs Analyzed and Includes Outcomes: Primary Non-Response, Secondary Loss of Response, and Time to Loss of Response

TABLE 3(A)
Table 3(A) depicts top IBD risk loci from
GWA studies and primary non-response.

| | Primary non | | | | | |
|---|---|---|---|---|---|---|
| | response | Genotype | | Genotype | | |
| SNP | Yes = 1 No = 0 | 12/22 | | 11 | | P Value |
| rs2476601 | 0 | 7 | (12.96) | 47 | (87.04) | 0.311 |
| | 1 | | | 7 | (100.00) | |
| rs2274910 | 0 | 23 | (42.59) | 31 | (57.41) | 0.150 |
| | 1 | 5 | (71.43) | 2 | (28.57) | |
| rs9286879 | 0 | 28 | (51.85) | 26 | (48.15) | 0.654 |
| | 1 | 3 | (42.86) | 4 | (57.14) | |
| rs2241880 | 0 | 30 | (56.82) | 21 | (41.18) | 0.04 |
| | 1 | 7 | (100.00) | | | |
| rs3197999 | 0 | 34 | (62.96) | 20 | (37.04) | 0.082 |
| | 1 | 2 | (28.57) | 5 | (71.43) | |
| rs4613763 | 0 | 4 | (7.41) | 50 | (92.59) | 0.533 |
| | 1 | 1 | (14.29) | 6 | (85.71) | |
| rs10044354 | 0 | 38 | (70.37) | 16 | (29.63) | 0.477 |
| | 1 | 4 | (57.14) | 3 | (42.86) | |
| rs2188962 | 0 | 45 | (63.33) | 9 | (16.67) | 0.03 |
| | 1 | 3 | (42.86) | 4 | (57.14) | |
| rs10045431 | 0 | 22 | (40.74) | 32 | (59.26) | 0.535 |
| | 1 | 2 | (28.57) | 5 | (71.43) | |
| rs6908425 | 0 | 13 | (24.07) | 41 | (75.93) | 0.066 |
| | 1 | 4 | (57.14) | 3 | (42.86) | |
| rs2844480 | 0 | 22 | (40.74) | 32 | (59.26) | 0.535 |
| | 1 | 2 | (28.57) | 5 | (71.43) | |
| rs2301436 | 0 | 36 | (66.67) | 18 | (33.33) | 0.618 |
| | 1 | 4 | (57.14) | 3 | (42.86) | |
| rs1456893 | 0 | 29 | (53.70) | 25 | (46.30) | 0.211 |
| | 1 | 2 | (28.57) | 5 | (71.43) | |
| rs1551398 | 0 | 32 | (59.26) | 22 | (40.74) | 0.535 |
| | 1 | 5 | (71.43) | 2 | (28.57) | |
| rs2456449 | 0 | 31 | (57.41) | 23 | (42.59) | 0.466 |
| | 1 | 3 | (42.86) | 4 | (57.14) | |
| rs10758669 | 0 | 43 | (79.63) | 11 | (20.37) | 0.618 |
| | 1 | 5 | (71.43) | 2 | (28.57) | |
| rs4574921 | 0 | 21 | (38.89) | 33 | (61.11) | 0.202 |
| | 1 | 1 | (14.29) | 6 | (85.71) | |
| rs10995239 | 0 | 35 | (64.81) | 19 | (35.19) | 0.259 |
| | 1 | 3 | (42.86) | 4 | (57.14) | |
| rs11190140 | 0 | 39 | (72.22) | 15 | (27.78) | 0.445 |
| | 1 | 6 | (85.71) | 1 | (14.29) | |
| rs3764147 | 0 | 31 | (57.41) | 23 | (42.59) | 0.004 |
| | 1 | | | 7 | (100.00) | |
| rs1968752 | 0 | 26 | (51.65) | 26 | (48.15) | 0.654 |
| | 1 | 3 | (42.86) | 4 | (57.14) | |
| rs8049439 | 0 | 25 | (46.30) | 29 | (53.70) | 0.864 |
| | 1 | 3 | (42.86) | 4 | (57.14) | |
| rs2076756 | 0 | 32 | (59.26) | 22 | (40.74) | 0.409 |
| | 1 | 3 | (42.86) | 4 | (57.14) | |
| rs2872507 | 0 | 41 | (75.93) | 13 | (24.07) | 0.143 |
| | 1 | 7 | (100.00) | | | |
| rs744166 | 0 | 27 | (50.00) | 27 | (50.00) | 0.285 |
| | 1 | 5 | (71.43) | 2 | (28.57) | |
| rs762421 | 0 | 34 | (62.96) | 20 | (37.04) | 0.03 |
| | 1 | 1 | (14.29) | 6 | (85.71) | |
| rs10489630 | 0 | 36 | (66.67) | 18 | (33.33) | 0.050 |
| | 1 | 2 | (28.57) | 5 | (71.43) | |
| rs917997 | 0 | 26 | (46.15) | 28 | (51.85) | 0.792 |
| | 1 | 3 | (42.86) | 4 | (57.14) | |
| rs9271568 | 0 | 31 | (58.49) | 22 | (41.51) | 0.004 |
| | 1 | | | 7 | (100.00) | |
| rs11174631 | 0 | 9 | (16.67) | 45 | (83.33) | 0.242 |
| | 1 | | | 7 | (100.00) | |
| rs991804 | 0 | 24 | (44.44) | 30 | (55.56) | 0.424 |
| | 1 | 2 | (28.57) | 5 | (71.43) | |
| rs2836878 | 0 | 29 | (53.70) | 25 | (46.30) | 0.01 |
| | 1 | | | 7 | (100.00) | |
| rs3749946 | 0 | 36 | (66.67) | 18 | (33.33) | 0.050 |
| | 1 | 2 | (28.57) | 5 | (71.43) | |
| rs7228236 | 0 | 26 | (48.15) | 28 | (51.85) | 0.792 |
| | 1 | 3 | (42.86) | 4 | (57.14) | |

TABLE 3(B)
Table 3(B) depicts top IBD loci from GWA
studies and secondary loss of response.

| | Secondary Loss | | | | | |
|---|---|---|---|---|---|---|
| | of response | genotype | | genotype | | |
| SNP | YES = 1, NO = 0 | 12/22 | | 11 | | P value |
| rs2476601 | 0 | 7 | (18.42) | 31 | (81.58) | 0.066 |
| | 1 | | | 16 | (100.00) | |
| rs2274910 | 0 | 16 | (42.11) | 22 | (57.89) | 0.911 |
| | 1 | 7 | (43.75) | 9 | (56.25) | |
| rs9266879 | 0 | 19 | (50.00) | 19 | (50.00) | 0.675 |
| | 1 | 9 | (56.25) | 7 | (43.75) | |
| rs2241880 | 0 | 22 | (57.89) | 16 | (42.11) | 0.818 |
| | 1 | 8 | (61.54) | 5 | (38.46) | |
| rs3197999 | 0 | 23 | (60.53) | 15 | (39.47) | 0.568 |
| | 1 | 11 | (68.75) | 5 | (31.25) | |
| rs4613763 | 0 | 4 | (10.53) | 34 | (89.47) | 0.177 |
| | 1 | | | 16 | (100.00) | |
| rs10044354 | 0 | 30 | (78.95) | 8 | (21.05) | 0.033 |
| | 1 | 8 | (50.00) | 8 | (50.00) | |
| rs2188962 | 0 | 31 | (81.58) | 7 | (18.42) | 0.594 |
| | 1 | 14 | (87.50) | 2 | (12.50) | |
| rs10045431 | 0 | 16 | (42.11) | 22 | (57.89) | 0.753 |
| | 1 | 6 | (37.50) | 10 | (62.50) | |
| rs6908425 | 0 | 6 | (15.79) | 32 | (84.21) | 0.028 |
| | 1 | 7 | (43.75) | 9 | (56.25) | |
| rs2844480 | 0 | 15 | (39.47) | 23 | (60.53) | 0.770 |
| | 1 | 7 | (43.75) | 9 | (56.25) | |
| rs2301436 | 0 | 26 | (68.42) | 12 | (31.58) | 0.673 |
| | 1 | 10 | (62.50) | 6 | (37.50) | |
| rs1456893 | 0 | 20 | (52.63) | 18 | (47.37) | 0.808 |
| | 1 | 9 | (56.25) | 7 | (43.75) | |
| rs1551398 | 0 | 23 | (60.53) | 15 | (39.47) | 0.770 |
| | 1 | 9 | (56.25) | 7 | (43.75) | |
| rs2456449 | 0 | 18 | (47.37) | 20 | (52.63) | 0.021 |
| | 1 | 13 | (81.25) | 3 | (18.75) | |
| rs10758669 | 0 | 31 | (61.58) | 7 | (18.42) | 0.584 |
| | 1 | 12 | (75.00) | 4 | (25.00) | |
| rs4674921 | 0 | 16 | (42.11) | 22 | (57.89) | 0.455 |
| | 1 | 5 | (31.25) | 11 | (68.75) | |
| rs10995239 | 0 | 27 | (71.05) | 11 | (28.95) | 0.139 |
| | 1 | 8 | (50.00) | 8 | (50.00) | |
| rs11190140 | 0 | 26 | (68.42) | 12 | (31.58) | 0.337 |
| | 1 | 13 | (81.25) | 3 | (18.75) | |
| rs3764147 | 0 | 22 | (57.89) | 16 | (42.11) | 0.911 |
| | 1 | 9 | (56.25) | 7 | (43.75) | |
| rs1968752 | 0 | 19 | (50.00) | 19 | (50.00) | 0.675 |
| | 1 | 9 | (56.25) | 7 | (43.75) | |
| rs8049439 | 0 | 14 | (36.84) | 24 | (63.16) | 0.032 |
| | 1 | 11 | (68.75) | 5 | (31.25) | |
| rs2076756 | 0 | 23 | (60.53) | 15 | (39.47) | 0.770 |
| | 1 | 9 | (56.25) | 7 | (43.75) | |
| rs2872507 | 0 | 30 | (78.95) | 8 | (21.05) | 0.424 |
| | 1 | 11 | (68.75) | 5 | (31.25) | |
| rs744166 | 0 | 19 | (50.00) | 19 | (50.00) | 1.000 |
| | 1 | 8 | (50.00) | 8 | (50.00) | |
| rs762421 | 0 | 24 | (63.16) | 14 | (36.84) | 0.964 |
| | 1 | 10 | (62.50) | 6 | (37.50) | |
| rs10489630 | 0 | 24 | (63.16) | 14 | (36.84) | 0.399 |
| | 1 | 12 | (75.00) | 4 | (25.00) | |
| rs917997 | 0 | 19 | (50.00) | 19 | (50.00) | 0.675 |
| | 1 | 7 | (43.75) | 9 | (56.25) | |
| rs9271568 | 0 | 22 | (59.46) | 15 | (40.54) | 0.828 |
| | 1 | 9 | (56.25) | 7 | (43.75) | |
| rs11174631 | 0 | 9 | (23.68) | 29 | (76.32) | 0.033 |
| | 1 | | | 16 | (100.00) | |
| rs991804 | 0 | 15 | (39.47) | 23 | (60.53) | 0.257 |
| | 1 | 9 | (86.25) | 7 | (43.75) | |
| rs2836878 | 0 | 19 | (50.00) | 19 | (50.00) | 0.400 |
| | 1 | 10 | (62.50) | 6 | (37.50) | |
| rs3749946 | 0 | 24 | (63.16) | 14 | (36.84) | 0.399 |
| | 1 | 12 | (75.00) | 4 | (25.00) | |
| rs7228236 | 0 | 19 | (50.00) | 19 | (50.00) | 0.675 |
| | 1 | 7 | (43.75) | 9 | (56.25) | |

TABLE 3(C)
Table 3(C) depicts time of loss of response: Kaplan Meier
Survival Analysis (time to loss of response).

| | | | mean | | | |
|---|---|---|---|---|---|---|
| SNP | genotype | number | duration | P | % censored | P |
| rs2476601 | 11 | 47 | 26.234 | 0.893 | 67% | 0.12 |
| | 12/22 | 7 | 25.000 | | 100% | |
| rs2274910 | 11 | 31 | 24.613 | 0.582 | 71% | 0.94 |
| | 12/22 | 23 | 28.043 | | 69.60% | |
| rs9286879 | 11 | 26 | 23.808 | 0.478 | 73.10% | 0.94 |
| | 12/22 | 28 | 28.179 | | 67.90% | |
| rs2241880 | 11 | 21 | 33.238 | 0.098 | 76.20% | 0.48 |
| | 12/22 | 30 | 22.567 | | 73.30% | |
| rs3197999 | 11 | 20 | 28.550 | 0.537 | 75% | 0.62 |
| | 12/22 | 34 | 24.618 | | 67.70% | |
| rs4613763 | 11 | 50 | 25.840 | 0.788 | 68% | 0.21 |
| | 12/22 | 4 | 29.000 | | 100% | |
| rs10044354 | 11 | 16 | 20.938 | 0.277 | 50% | 0.02 |
| | 12/22 | 38 | 28.237 | | 78.90% | |
| rs2188962 | 11 | 9 | 21.556 | 0.511 | 77.80% | 0.67 |
| | 12/22 | 45 | 26.978 | | 68.90% | |
| rs10045431 | 11 | 32 | 25.031 | 0.683 | 68.80% | 0.66 |
| | 12/22 | 22 | 27.591 | | 72.70% | |
| rs6908425 | 11 | 41 | 27.122 | 0.545 | 78.10% | 0.04 |
| | 12/22 | 13 | 22.769 | | 46.20% | |
| rs2844480 | 11 | 32 | 23.625 | 0.336 | 71.90% | 0.95 |
| | 12/22 | 22 | 29.636 | | 68.20% | |
| rs2301436 | 11 | 18 | 24.222 | 0.671 | 66.70% | 0.62 |
| | 12/22 | 36 | 27.000 | | 72.20% | |
| rs1456893 | 11 | 25 | 27.200 | 0.734 | 72% | 0.94 |
| | 12/22 | 29 | 25.103 | | 69% | |
| rs1551398 | 11 | 22 | 28.818 | 0.459 | 68.20% | 0.99 |
| | 12/22 | 32 | 24.188 | | 71.90% | |
| rs2456449 | 11 | 23 | 26.913 | 0.815 | 87% | 0.03 |
| | 12/22 | 31 | 25.452 | | 58.10% | |
| rs10758669 | 11 | 11 | 20.455 | 0.355 | 64.60% | 0.28 |
| | 12/22 | 43 | 27.512 | | 72.10% | |
| rs4574921 | 11 | 33 | 25.061 | 0.680 | 66.70% | 0.49 |
| | 12/22 | 21 | 27.667 | | 76.20% | |
| rs10995239 | 11 | 19 | 24.105 | 0.638 | 57.90% | 0.16 |
| | 12/22 | 35 | 27.143 | | 77.10% | |
| rs11190140 | 11 | 15 | 31.267 | 0.294 | 80% | 0.25 |
| | 12/22 | 39 | 24.077 | | 66.70% | |
| rs3764147 | 11 | 23 | 26.522 | 0.900 | 69.60% | 0.93 |
| | 12/22 | 31 | 25.742 | | 71% | |
| rs1968752 | 11 | 26 | 19.231 | 0.029 | 73.10% | 0.86 |
| | 12/22 | 28 | 32.429 | | 67.90% | |
| rs8049439 | 11 | 29 | 27.000 | 0.746 | 82.80% | 0.07 |
| | 12/22 | 25 | 25.000 | | 56% | |
| rs2076756 | 11 | 22 | 26.909 | 0.822 | 68.20% | 0.89 |
| | 12/22 | 32 | 25.500 | | 71.90% | |
| rs2872507 | 11 | 13 | 31.462 | 0.323 | 61.50% | 0.63 |
| | 12/22 | 41 | 24.366 | | 73.20% | |
| rs744166 | 11 | 27 | 21.519 | 0.135 | 70.40% | 0.68 |
| | 12/22 | 27 | 30.630 | | 70.40% | |
| rs762421 | 11 | 20 | 22.900 | 0.428 | 70% | 0.89 |
| | 12/22 | 34 | 27.941 | | 70.60% | |
| rs10489630 | 11 | 18 | 31.556 | 0.205 | 77.90% | 0.42 |
| | 12/22 | 36 | 23.333 | | 66.70% | |
| rs917997 | 11 | 28 | 27.571 | 0.614 | 67.90% | 0.57 |
| | 12/22 | 26 | 24.462 | | 73.10% | |
| rs9271568 | 11 | 22 | 30.045 | 0.340 | 68.20% | 0.99 |
| | 12/22 | 31 | 24.065 | | 71% | |
| rs11174631 | 11 | 45 | 22.622 | 0.010 | 64.40% | 0.03 |
| | 12/22 | 9 | 43.333 | | 100% | |
| rs991804 | 11 | 30 | 27.500 | 0.605 | 76.70% | 0.24 |
| | 12/22 | 24 | 24.292 | | 62.50% | |
| rs2836878 | 11 | 25 | 29.000 | 0.376 | 76% | 0.42 |
| | 12/22 | 29 | 23.552 | | 65.50% | |
| rs3749946 | 11 | 18 | 31.556 | 0.205 | 77.80% | 0.42 |
| | 12/22 | 36 | 23.333 | | 66.70% | |
| rs7228236 | 11 | 28 | 27.571 | 0.614 | 67.90% | 0.57 |
| | 12/22 | 26 | 24.462 | | 73.10% | |

TABLE 3(D)
Table 3(D) depicts serological immune markers and
anti-TNFα responsiveness (n = 63).

| Immune | Positive = 1 | | | | | |
|---|---|---|---|---|---|---|
| Response | Negative = 0 | yes | no | P | OR | 95 % CI |
| | | Primary non response | | | | |
| pANCA | 1 | 6(85.71) | 12(25.53) | 0.002 | 17.5 | 1.91-160.5 |
| | 0 | 1(14.29) | 35(74.47) | | | |
| ASCA IgA | 1 | | 30(47.62) | 0.004 | 0.05 | |
| and/or IgG | | | | | | |
| | 0 | 10(100.00) | 33(52.38) | | | |
| OmpC | 1 | 1(10.00) | 16(25.40) | 0.29 | 0.33 | 0.04-2.78 |
| | 0 | 9(90.00) | 47(74.60) | | | |
| I2 | 1 | 3(30.00) | 19(30.65) | 0.97 | 0.97 | 0.23-4.16 |
| | 0 | 7(70.00) | 43(69.35) | | | |
| CBir1 | 1 | 5(50.00) | 28(45.16) | 0.78 | 1.20 | 0.32-4.62 |
| | 0 | 5(50.00) | 34(54.84) | | | |
| | | Secondary Loss of response | | | | |
| pANCA | 1 | 4(36.36) | 8(22.22) | 0.35 | 2.000 | 0.47-8.60 |
| | 0 | 7(63.64) | 28(77.78) | | | |
| ASCA IgA | 1 | 6(33.33) | 24(53.33) | 0.15 | 0.438 | 0.14-1.37 |
| and/or IgG | | | | | | |
| | 0 | 12(66.67) | 21(46.67) | | | |
| OmpC | 1 | 6(33.33) | 10(22.22) | 0.36 | 1.750 | 0.52-5.85 |
| | 0 | 12(66.67) | 35(77.78) | | | |
| I2 | 1 | 8(44.44) | 11(25.00) | 0.13 | 2.400 | 0.76-7.60 |
| | 0 | 10(55.56) | 33(75.00) | | | |
| CBir1 | 1 | 7(38.89) | 21(47.73) | 0.53 | 0.697 | 0.23-2.13 |
| | 0 | 11(61.11) | 23(52.27) | | | |

### Example 8

### Enrollment Criteria

### Inclusion Criteria:

1. IBD patients <21 years of age
2. Active CD or UC patients initiating infliximab therapy as prescribed by the treating physician.
3. No predetermined minimum Crohn's Disease Activity Index (CDAI) or Pediatric Crohn's Disease Activity Index (PCDAI) or Partial Mayo score will be necessary for inclusion (see justification below)
4. Willingness to participate
5. Able to give consent by patient or legal guardian
6. Willing to provide blood

### Exclusion Criteria:

1. IBD Patients not receiving infliximab
2. Patients in remission at time of initiating infliximab
3. IBD patients who are on low dose prednisone for adrenal insufficiency and unable to wean in designated time frame.
4. CD patients with exclusive perianal fistulizing disease (see justification below)

### Example 9

### Association of Anti-TNF Responsiveness Against the Whole Genome

Three (3) outcomes were evaluated:
1) primary non-response: patient did not respond to the induction regimen as defined by patient did not receive a clinical benefit from the first 3 infusions of infliximab and did not receive any further treatment doses.
2) secondary loss of response: patient responded to the induction regimen and despite dose escalation and/or frequency intensification of infliximab the drug was discontinued as of last follow up.
3) failure for any reason.

The results and analysis of the three outcomes are further described in Tables 1(A-C) and 2 (A-C) below.

### Example 10

### Table 4 (A-C)-Results of Top Ten (10) Most Significant Associations

TABLE 4(A-C)
Results of top ten (10) most significant associations
Analysis of top ten (10) most significant associations for
primary non-response as an outcome.

| snp | CHR | BP | A1 | F_A | F_U | A2 | fisher_p | OR | l_p | loci |
|---|---|---|---|---|---|---|---|---|---|---|
| rs13079040 | 3 | 68988334 | C | 0.7143 | 0.1574 | T | 0.00003361 | 13.38 | 4.47353149 | FAM19A4 |
| rs4855535 | 3 | 69017124 | G | 0.5714 | 0.05556 | T | 6.987E−06 | 22.67 | 5.15570926 | FAM19A4 |
| rs17048128 | 3 | 69028502 | A | 0.5714 | 0.05556 | G | 6.987E−06 | 22.67 | 5.15570926 | FAM19A4 |
| rs17048129 | 3 | 69031452 | A | 0.5714 | 0.05556 | G | 6.987E−06 | 22.67 | 5.15570926 | FAM19A4 |
| rs17039556 | 4 | 161545059 | A | 0.7143 | 0.1296 | G | 8.744E−06 | 16.79 | 5.05828985 | |
| rs12640159 | 4 | 161586073 | A | 0.7857 | 0.1944 | G | 0.00001882 | 15.19 | 4.72538038 | |
| rs880330 | 7 | 67842575 | T | 0.7857 | 0.213 | C | 0.00003927 | 13.55 | 4.4059391 | |
| rs2057917 | 7 | 67867804 | C | 0.8571 | 0.2778 | T | 0.00004331 | 15.6 | 4.36341182 | |
| rs2983478 | 14 | 94433843 | C | 1 | 0.4167 | T | 0.00001555 | | 4.80826961 | |
| rs4776127 | 15 | 51373815 | G | 0.7857 | 0.213 | A | 0.00003927 | 13.55 | 4.4059391 | |

TABLE 4(B)
Analysis of top ten (10) most significant associations for secondary loss of
response as an outcome.

| snp | CHR | BP | A1 | F_A | F_U | A2 | fisher_p | OR | l_p | loci |
|---|---|---|---|---|---|---|---|---|---|---|
| rs1838990 | 3 | 63142891 | C | 0.09375 | 0.5132 | T | 0.00002815 | 0.09814 | 4.5505216 | |
| rs11713998 | 3 | 168613099 | T | 0.4375 | 0.06579 | C | 0.00001437 | 11.04 | 4.84254323 | |
| rs13144587 | 4 | 67295867 | C | 0.4375 | 0.07895 | A | 0.00003895 | 9.074 | 4.40949254 | |
| rs6928719 | 6 | 166117879 | C | 0.1875 | 0.6711 | A | 5.292E−06 | 0.1131 | 5.27638016 | |
| rs6928737 | 6 | 166118611 | G | 0.1875 | 0.6579 | A | 7.617E−06 | 0.12 | 5.11821604 | |
| rs6904237 | 6 | 166121014 | C | 0.1875 | 0.6316 | T | 0.00004141 | 0.1346 | 4.38289477 | |
| rs4762507 | 12 | 97659769 | T | 0 | 0.3553 | C | 0.0000171 | 0 | 4.76700389 | ANKS1B |
| rs12857230 | 13 | 42654650 | G | 0.7188 | 0.25 | T | 8.917E−06 | 7.667 | 5.04978123 | |
| rs12918939 | 16 | 64698060 | G | 0.4375 | 0.07895 | A | 0.00003895 | 9.074 | 4.40949254 | |
| rs5999636 | 22 | 33661041 | T | 0 | 0.3289 | C | 0.00004165 | 0 | 4.38038499 | |

TABLE 4(C)
Analysis of top ten (10) most significant associations for failure for any
reason as an outcome.

| snp | CHR | BP | A1 | F_A | F_U | A2 | fisher_p | OR | l_p | loci |
|---|---|---|---|---|---|---|---|---|---|---|
| rs11695174 | 2 | 9705766 | T | 0.2955 | 0.02564 | C | 0.00002682 | 15.94 | 4.57154123 | |
| rs17020744 | 2 | 81845572 | A | 0.25 | 0.01282 | G | 0.00004771 | 25.67 | 4.32139058 | |
| rs1549599 | 3 | 37506369 | G | 0.6818 | 0.2692 | A | 0.00001852 | 5.816 | 4.73235902 | ITGA9 |
| rs1569091 | 7 | 94356064 | A | 0.6136 | 0.2308 | G | 0.00003676 | 5.294 | 4.4346245 | |
| rs7335910 | 13 | 24220725 | G | 0.09091 | 0.4615 | A | 0.00001775 | 0.1167 | 4.75080164 | |
| rs4326996 | 15 | 61567335 | G | 0.25 | 0.01282 | T | 0.00004771 | 25.67 | 4.32139058 | |
| rs1554983 | 16 | 77425340 | A | 0.3182 | 0.7179 | G | 0.00002387 | 0.1833 | 4.62214758 | |
| rs9915945 | 17 | 6089542 | A | 0.3636 | 0.05128 | G | 0.00001539 | 10.57 | 4.81276138 | |
| rs4794558 | 17 | 50702952 | A | 0.5682 | 0.1923 | G | 0.00004192 | 5.526 | 4.37757873 | HLF |
| rs323391 | 17 | 69199193 | A | 0.4773 | 0.1154 | C | 0.00001732 | 7 | 4.76145211 | |

### Example 11

### Methods—Patient Population

94 pediatric CD and UC patients (age <21 years), followed at CSMC. Each received at least 2 doses (week 0 and 2) of infliximab. Clinical data was collected and stored in a secure database at CSMC. Serological immune responses (ELISA EU/ml) collected and analyzed at CSMC. Specifically, ASCA (IgG and IgA), anti-Cbir1 (IgG), PANCA (IgG) I2 (IgA), OmpC (IgA). Genotype data was also obtained.

### Example 12

### Methods—Primary Outcome Definitions

Primary non-response was evaluated for patients. For Crohn's Disease, primary non response was defined by no change or increase in HBI from baseline at week 10 or earlier if drug discontinued before week 6 infusion. For ulcerative colitis, primary non response was defined by no change or increase from baseline in the sum of stool frequency and rectal bleeding subscores of the partial Mayo score and no improvement in the physician's global assessment subscore.

### Example 13

### Methods—Association Variables

SNP selection of known IBD susceptibility SNPs (GWA significance) were taken from previous adult CD meta GWAS (32 loci), adult UC GWAS (5 loci), and pediatric IBD GWAS (2 loci). SNP selection was also taken of top 10 significant SNPs from pharmacogenetic GWAS, using chi square test. Serology status, of positive or negative, was determined with chi square test. Finally, demographic and phenotype data was taken with t-test for continuous variables and chi-square test for categorical variables.

### Example 14

### Methods—Predictive Models

Predictive models of non-response were created using Strategy A, B and C. Strategy A utilizes known susceptibility IBD SNPs, using logistic multiple regression, and stepwise selection of phenotype, serologic and genetic variables with univariate association p<0.1. Strategy B utilizes the top 10 SNPs from pharmacogenetics GWAS (p<2*10⁻⁵), using logistic multiple regression, and stepwise selection of phenotype, serologic and genetic variables with univariate association p<0.1. Strategy C is the combination of Strategy A and B (also known as Final Model).

With regard to clinical utility, the sensitivity, specificity, accuracy, ROC, positive likelihood ratio calculated for the models.

### Example 15

### Table 5—Results of Demographic and Phenotype Associations with Primary Non Response

From 94 patients meeting inclusion criteria, 22 exhibited primary non response, and 72 exhibited primary response.

TABLE 5

| | NON | | |
|---|---|---|---|
| CLINICAL VARIABLE | RESPONSE | RESPONSE | P Value |
| UC vs. CD | 14:8 | 6:66 | <0.0001 |
| Mean age at diagnosis (years) | 10.1 | 10.5 | 0.71 |
| Gender M:F | 8:14 | 40:32 | 0.12 |
| Disease duration at start of | 26 | 25 | 0.96 |
| IFX (months) | | | |
| IMM use at start of IFX (%) | 79% | 92.8% | 0.1 |
| Duration of IMM at start of | 12.7 | 15.3 | 0.57 |
| IFX (months) | | | |
| pANCA+ | 76.2% | 29% | 0.0001 |
| ASCA+ | 0% | 46.9% | 0.0003 |

### Example 16

### Table 6—Results of Strategy A: Models of Primary Non-Response with Known Susceptibility Loci

TABLE 6

| Model | Variable | Description | P value | OR (95% CI) | R-square |
|---|---|---|---|---|---|
| I | Diagnosis | UC vs. CD | 0.0001 | 15.0 | 0.48 |
| | | | | (4.1, 55.8) | |
| | pANCA | Pos vs. Neg | 0.01 | 5.4 | |
| | | | | (1.5, 19.9) | |
| II (known IBD | rs2188962 | CC vs. CT/TT | 0.04 | 3.3 | 0.30 |
| susceptibility | 5q31.1 | | | (1.03, 10.9) | |
| SNPs only) | | | | | |
| | rs6908425 | CT/TT vs. CC | 0.04 | 3.2 | |
| | 6p22.3 | | | (1.01, 10.4) | |
| | CDKA1 | | | | |
| | rs2836878 | GG vs AG/AA | 0.05 | 3.3 | |
| | 21q22.2/ | | | (0.98, 11.2) | |
| | BRWD1 | | | | |
| | rs2395185 | GG vs. TG/TT | 0.01 | 4.6 | |
| | 6p21/HLA-DQA1 | | | (1.4, 14.9) | |

### Example 17

### Table 7—Results of Strategy A: Models of Primary Non-Response with Known Susceptibility Loci

TABLE 7

| Model | Variable | Description | P value | OR (95% CI) | R-square |
|---|---|---|---|---|---|
| III: | Diagnosis | UC vs. CD | 0.0008 | 14.7 | 0.59 |
| I & II | | | | (3.1, 70.4) | |
| (Combined) | | | | | |
| | pANCA | Pos vs. Neg | 0.004 | 15.3 | |
| | | | | (2.4, 96.2) | |
| | rs6908425 | CT/TT vs. CC | 0.05 | 4.6 | |
| | 6p22.3 | | | (1.0, 21.0) | |
| | CDKA1 | | | | |
| | rs2836878 | GG vs. AG/AA | 0.02 | 9.8 | |
| | 21q22.2/ | | | (1.5, 64.0) | |
| | BRWD1 | | | | |
| | rs2395185 | GG vs. TG/TT | 0.047 | 5.4 | |
| | 6p21/HLA-DQA1 | | | (1.0, 28.6) | |

### Example 18

### Table 8—Results of Strategy B: Model of Primary Non-Response with Pharmacogenetic GWAS Loci

TABLE 8

| Model | Variable | Description | P Value | R square |
|---|---|---|---|---|
| IV | rs975664 | TT vs. CT/CC | 0.0006 | 0.67 |
| (pharmacogenetic | 2p12 | | | |
| GWAS genes only) | TACR1 | | | |
| | rs4855535 | GG/GT vs. TT | 0.006 | |
| | 3p14 | | | |
| | FAM19A4 | | | |
| | Rs4796606 | CC/CT vs. TT | 0.01 | |
| | 17q21 | | | |
| | KRT35, | | | |
| | 32, 36, 13 | | | |
| | Keratin gene | | | |
| | cluster | | | |
| | Rs765132 | TT/TC vs. CC | 0.03 | |
| | Xq26 | | | |

### Example 19

### Results—Strategy C: Final Model of Primary Non-Response with Pharmacogenetic GWA and Known Susceptibility SNPs and Clinical Phenotype and Serology Status

TABLE 9

| Model | Variable | Description | P value | R-square |
|---|---|---|---|---|
| V: | Dx | UC vs. CD | 0.008 | 0.82 |
| III & IV | pANCA | Pos vs. Neg | 0.03 | |
| | rs975664 | TT vs. CT/CC | 0.01 | |
| | 2p12/TACR1 | | | |
| | rs4855535 | GG/GT vs. TT | 0.02 | |
| | 3p14/FAM19A4 | | | |
| | rs6100556 | TG/TT vs. GG | 0.02 | |
| | 20q13/PHACTR3 | | | |
| | rs2836878 | GG vs. AG/AA | 0.07 | |
| | 21q22/BRWD1 | | | |

### Example 20

### Table 10—Results Demonstrating Diagnostic Utility of Final Model for Non-Response to Anti-TNFα

TABLE 10

| | | | | | | | Positive |
|---|---|---|---|---|---|---|---|
| | | | | | | | Likelihood |
| Model | Variable | Description | ROC | SN | SP | ACC | Ratio of NR |
| V: | Dx | UC vs. CD | 0.98 | 0.95 | 0.88 | 0.92 | 8 |
| III & IV | pANCA | Pos vs. Neg | | (cut-off: ≥ 3 | | | |
| | rs975664 | TT vs CT/CC | | risk factors | | | |
| | 2p12/ | | | | | | |
| | TACR1 | | | | | | |
| | rs4855535 | GG/GT vs TT | | | | | |
| | 3p14/ | | | | | | |
| | FAM19A4 | | | | | | |
| | rs6100556 | TG/TT vs GG | | | | | |
| | 20q13/ | | | | | | |
| | PHACTR3 | | | | | | |
| | rs2836878 | GG vs AG/AA | | | | | |
| | 21q22/ | | | | | | |
| | BRWD1 | | | | | | |

SN = sensitivity;
SP = specificity;
ACC = accuracy.

### Example 21

### Conclusions

4 known IBD susceptibility genes were associated with non-response to infliximab.
UC-independent predictor of non-response to infliximab and may be the most important predictor.
pANCA positive independently predicts non-response to infliximab.
Pharmacogenetic GWAS top loci improved prediction of non-response compared to known susceptibility loci.
The combination of genotype, phenotype and serotype was best predictor of non-response to anti-TNFα.
Defining predictors of response to anti-TNFα aid clinicians in choosing the right therapy for the right patient.
Trial design may be enhanced using genetic markers.

### Example 22

### Genotyping

Genotyping may be performed at Cedars-Sinai Medical Center in the GCRC phenotyping/genotyping Core Facility. Genotyping for single nucleotide polymorphisms (SNPs) is performed using ABI TaqMan MGB chemistry with an ABI 7900 instrument in 384 well format. A robotic workstation (Tecan Genesis) is used to set up the PCR plates and the current throughput is 6×384 assays per day. TaqMan chemistry results in the release of dye as the 5′-nuclease activity of Taq polymerase degrades a probe/quencher bound to a particular allele. The use of two dyes, one for each allele, allows the SNP genotyping in a single PCR reaction. The “MGB” system has several advantages over earlier version of this chemistry for SNP genotyping and is therefore a “second generation” technology that includes: 1) a more sensitive VIC dye rather than TET, 2) an improved optical system in the ABI7900 instrument eliminates missed reads due to scratches in the caps, 3) primer and probe design software that considerably lowers the amount of trouble-shooting 4) 384-well format for high throughput, and 5) use of a minor groove binder (MGB) shortens the length of the probe and thus increases discrimination between the two alleles of the SNP. This technology has been used extensively in the CSMC genotyping core.

### Example 23

### Definitions of Therapeutic Responsiveness

Response criteria described below have been employed in phase 3 clinical trials in both pediatric and adult IBD patients receiving infliximab. It is important to determine whether a patient is responding to their induction regimen. This will negate any non responders from continuing to receive ineffective therapy. The primary goal of using anti-TNFα is to induce and maintain a steroid free remission. The side effect profile and the potential growth stunting effects of corticosteroids make this steroid sparing agent very important in IBD patients, especially children. Having a strict corticosteroid specification for response makes for a more robust end point and potentially more clinically meaningful.

### Primary Non-Response:

CD: Inability to achieve a drop in PCDAI of >15 points from baseline or CDAI drop of >70 points and >75% reduction in corticosteroid dose at week 10 if on steroids at initiation of infliximab.
UC: Inability to achieve an improvement of at least 2 points in the sum of stool frequency and rectal bleeding subscores of the partial Mayo score, and no worsening in the physician's global assessment subscore OR the sum of the subject's stool frequency and rectal bleeding subscores is less than or equal to 1, and no worsening in the physician's global assessment subscore and >75% reduction in corticosteroid dose at week 10 if on corticosteroids at initiation of infliximab.

### Response:

CD: Drop in PCDAI of >15 points from baseline or CDAI drop of >70 points from baseline for CD and >75% reduction in corticosteroid dose at week 10 if on corticosteroids at initiation of infliximab. For example a patient started on 40 mg/day will need to be weaned to <10 mg/day.
UC: An improvement of at least 2 points in the sum of stool frequency and rectal bleeding subscores of the partial Mayo score, and no worsening in the physician's global assessment subscore OR the sum of the subject's stool frequency and rectal bleeding subscores is less than or equal to 1, and no worsening in the physician's global assessment subscore and >75% reduction in corticosteroid dose at week 10 if on corticosteroids at initiation of infliximab.

### Forced Corticosteroid Taper: (Standard Corticosteroid Wean Based on Clinical Trials and Routine Clinical Care);

Subjects on oral prednisone or equivalent will keep their enrollment dose stable for 2 weeks as anti-TNFα may take 10-14 days to have a clinical effect. Starting at week 2, corticosteroids will be tapered at the following rate:

Prednisone or equivalent: >20 mg/day 5 mg/q 5 days
>10−<20 mg/day 5 mg/week
<10 mg/day 2.5 mg/week

### Sustained Remission:

CD: PCDAI<10 or CDAI<150 for CD and off corticosteroids at week 10 and week 54 without rescue therapy with steroids or infliximab dose increase or frequency escalation in between.
UC: partial mayo score ≤2 points, with no individual subscore >1 and off corticosteroids at week 10 and week 54 without rescue therapy with steroids or infliximab dose increase or frequency escalation in between.

### Loss of Response:

Recurrence of symptoms as determined by the treating physician necessitating rescue therapy with corticosteroids or an increase dose of infliximab from 5 mg/kg to 10 mg/kg or the patient requires infliximab more frequent then every 8 weeks. Physician Global Assessment Score will be used to document the condition of the patient at time of the visit determining loss of response and change in clinical condition from week 10 assessment. The physician global assessment of change is a 5-point scale used to assess the change from baseline (week 10) in the subject's disease activity from the perspective of the physician.

### Immunogenicity and Infliximab Trough Drug Levels:

Immunogenicity may be determined based on the development of antibodies to infliximab. Antibody levels will be measured as detectable vs. non detectable. Levels >8 ug/dl have been shown to be associated with loss of response and will be chosen as cut point for detectable vs. non detectable.

Trough drug levels: levels of infliximab in the blood at the time of the infliximab infusion. Levels <12 μg/ml have been shown to be associated with loss of response and will be chosen as threshold for detectable vs. non detectable.

mRNA Expression:

Subject's mRNA expression may be classified as respondents or non-respondents. Respondents will be determined by a more than 2-fold increase in mRNA expression after induction.

### Clinical Phenotype:

The baseline clinical features to be analyzed include: age at diagnosis, gender, disease location (upper tract, small bowel, large bowel, perianal), disease behavior (fistulizing and or stricturing disease), duration of disease at baseline, disease activity at baseline, smoking (active or passive) history and use of concomitant immunomodulators.

### Example 24

### Serological Immune Response Processing

### ASCA/ANCA ELISA:

ASCA: the samples will be analyzed by ELISA with phosphopeptidomannan extracted from Saccharomyces cerevisiae serving as the antigen. Briefly, plasma diluted 1:80 (for IgA detection) or 1:800 (for IgG detection) will be added to plates previously coated with mannan at 100 ug/ml. After incubation and washing, alkaline phosphatase labeled goat anti-human IgA and IgG are added to their respective plates. Finally, after another incubation and wash, substrate (p-nitrophenyl phosphate) is added and color change is detected at 405 nm. All samples are compared to standard positive control samples and expressed as ELISA units (EU).

ANCA/pANCA: the samples will be quantitatively analyzed by ELISA and ELISA positive samples will be further characterized qualitatively by immunofluorescence binding pattern including DNase treatment of samples with pANCA binding. For ELISA analysis, microtiter plates are coated with a monolayer of neutrophils (25,000/well) and air-dried, fixed with 100% methanol, dried again and stored at −20° C. For use, the plates are blocked for non specific binding by 0.5% bovine serum albumin in phosphate buffered saline (BSA/PBS), the blocking material is discarded and samples at a 1:100 dilution in BSA/PBS are added. After incubation and washing, alkaline phosphatase labeled goat anti-human IgG (gamma chain specific) is added. Finally, after another incubation and wash, substrate (p-nitrophenyl phosphate) is added and color change is detected at 405 nm. All samples are compared to standard positive control samples and expressed as ELISA units (EU). For indirect immunofluorescence analysis of ANCA, slides are prepared by cytocentrifugation of 100,000 neutrophils. The slides are air-dried and fixed in 100% methanol, then air-dried and stored at −20° C. For use, the slides are rehydrated in PBS and samples diluted 1:20 in BSA/PBS are added. After washing, fluoroscein labeled goat F (ab)′2 anti human IgG (gamma chain specific) is added. The slides are washed again and evaluated by fluorescence microscopy. For evaluation of DNase sensitivity of pANCA patterns, slides are pretreated with 100 U/ml of RNase free DNase for 30 minutes. Slides are stained as above with a pair of DNase-treated and untreated slides used for each sample.

### CBir1 ELISA:

ELISA analysis of anti-CBir1 was performed as previously described 21 but using NH2-terminal fragment of CBir1 (147aa) without knowledge of diagnosis or other serology results. Briefly, ELISA plates were coated overnight with 100 ng/well of CBir1, then blocked with 1% BSA in PBS for 2 hours. Plates were washed and serum was added at a 1:200 dilution in 1% BSA-PBS for a 30 minute incubation. After washing, horseradish peroxidase conjugated anti-human IgG at a 1:10,000 dilution was added and incubated for 30 minutes. After another wash, the plates were incubated with tetramethylbenzidine substrate for 15 minutes. The reaction was stopped with 1 N sulfuric acid and read at 450 nm. Positive was defined as the mean+2 SD of the healthy controls. For Cohort 2 and the longitudinal cohorts and phenotype cohorts, this assay was modified to be more similar to the ANCA, OmpC and 12 protocols: alkaline phosphatase was substituted as the secondary conjugate and incubated for 1 hour followed by paranitrophenyl phosphate as substrate for 30 minutes.

### OmpC/I2 Purification

Trimeric OmpC is biochemically purified from an OmpF−/−/OmpA−/− disruptive insertion mutant E. coli K12 (provided by R. Misra). Mutant E. coli glycerol stocks are inoculated into 10-20 ml of Luria Bertani broth supplemented with 100 ug/ml Streptomycin (LB-Strep), and culture vigorously at 37° C. for ˜8 hours to log phase followed by expansion to 1 liter in LB-Strep over 15 hours at 25° C. Cells are harvested by centrifugation, washed twice with 100 ml of ice cold 20 mM Tris-Cl pH 7.5, and resuspend in cold spheroplast forming buffer (20 mM Tris-Cl pH 7.5, 20% Sucrose, 0.1M EDTA pH 8.0, 1 mg/ml Lysozyme). Spheroplasts are allowed to form for 1 hour on ice with occasional mixing, and then lysed by 14 fold dilution into ice cold 10 mM Tris-Cl pH 7.5, 1 mg/ml DNase-I, and vigorous vortexing followed by pulse sonication (4×30 seconds, On time=1 second at high power). Cell debris is by low speed centrifugation, and membrane preparation collected by ultra centrifugation at 100,000 g in a swing bucket rotor. Membrane pellet is resuspended by homogenizing into 20 mM Tris-Cl pH 7.5, and extracted for 1 hour in 20 mM Tris-Cl pH 7.5+1% SDS by rotating at 37° C. Pre-extracted membrane preparation is then pelleted by ultracentrifugation and resuspended by homogenizing into 20 mM Tris-Cl pH 7.5 as above, and OmpC is extracted for 1 hour rotating at 37° C. with 20 mM Tris-Cl pH 7.5, 3% SDS, and 0.5M NaCl. Membrane is then pelleted by ultracentrifugation and the supernatant containing trimeric OmpC is collected. SDS is removed from OmpC preparations by detergent exchange dialysis against >10,000 volumes of 0.2% triton x100 followed by dialysis against >10,000 volumes Tris-Cl pH 7.5. Purified OmpC is quantified using the Bradford reagent (Biorad, Hercules, CA) and purity of >95% is validated by SDS-PAGE and Silver staining (Biorad). Purified protein is aliquoted and stored at −20° C. until used.

The 100 amino acid open reading frame (ORF) of 12 is subcloned into pGEX-KG and expressed in E. coli XL-1 blue (Stratagene, La Jolla, CA). 12-GST fusion protein is present as an inclusion body, and purified according to manufacturer's instructions by differential solubilization in 0.1% sodium dodecyl sulfate (SDS). The glutathione-S-transferase (GST) control is produced with unmodified pGEX-KG and XL-1 blue cells, and is present about 50% in the soluble and inclusion body fractions. The latter is purified exactly as 12-GST, and the former is purified by G-Sepharose affinity chromatography. All protein preparations are >90% pure by SDS polyacrylamide gel electrophoresis (PAGE) and Coomassie blue protein staining.

### Determination and Characterization of the OmpC/12 Response:

Human IgA antibodies that bind I2 or OmpC will be detected by direct ELISA assays. Plates (Greiner, USA Scientific, Ocala, FL) will be coated overnight at 4 C with 100 μl/well of GST alone and I2-GST (5 μg/ml) or OmpC (0.25 μg/ml) in borate buffered saline, pH 8.5. After three washes in 0.05% Tween 20 in phosphate buffered saline (PBS), the plates will be blocked with 150 μl/well of 0.5% bovine serum albumin in PBS, pH 7.4 (BSA-PBS) for 30 minutes at room temperature (RT). The blocking solution will then be discarded and 100 μl/well of sera diluted 1:100 will be added and incubated for 2 hours at RT. The plates will be washed as before and alkaline phosphatase conjugated goat anti-human IgA (_-chain specific, Jackson ImmunoResearch, West Grove, PA) at a dilution of 1:1000 in BSA-PBS will be added for 2 hours at RT. The plates will be washed three times with 0.05% Tween 20 in phosphate buffered saline followed by another three washes with Tris buffered normal saline, pH7.5. Substrate solution (1.5 mg/ml disodium P-nitrophenol phosphate (Amresco, Solon, OH), 2.5 mM MgCl2, 0.01M Tris, pH 8.6) will be added at 100 _l/well and color will be allowed to develop for one hour at which time the plates will be read at 405 nm. Nonspecific binding of sera to GST alone (typically <0.1) will be subtracted from raw values of 12-GST binding to obtain 12 specific absorbances. Levels will be determined relative to a standard consisting of serum obtained from a well-characterized CD patient. Results will be expressed as ELISA units (EU/ml). Sera with antibody levels exceeding the normal reference range value will be termed positive.

### Example 25

### Blood Stimulation

Anti-TCR antibody (IgG1) or controls (control IgG1 BioLegend, San Diego, CA) may be stored at −20° C. in 6-well strip microtubes until use. Sixty microliters of whole blood will be added into 6 wells of microwell strip (3 wells for control IgG and 3 wells for anti-TCR antibody) and incubated at 37° C. for 4 hours with the cap closed. Following each treatment, blood samples were stored frozen at _80° C.

### Example 26

### Final Predictive Model-Overall

Inter-individual variation in response to anti-TNFα therapy may be explained by genetic variability in disease pathogenesis or mechanism of action. Recent genome wide association studies (GWAS) in IBD have increased understanding of the genetic susceptibility to IBD.

As disclosed herein, the inventors tested associations of known IBD susceptibility loci and novel “pharmacogenetic” GWAS identified loci with primary non-response to anti-TNFα in pediatric IBD patients and developed a predictive model of primary non-response. Primary non response was defined using the HBI for CD and partial Mayo score for UC. Genotyping was performed using the Illumina Infinium platform. Chi square analysis tested associations of phenotype and genotype with primary non-response. Genetic associations were identified by testing known IBD susceptibility loci and by performing a GWAS for primary non-response. Step-wise multiple logistic regression was performed to build predictive models.

As further disclosed herein, non-response occurred in 22 of 94 subjects. Six known susceptibility loci were associated with primary non-response (p<0.05). The 21q22.2/BRWD/loci remained significant in the predictive model. The most predictive model included 3 novel “pharmacogenetic” GWAS loci, the previously identified BRWD1, PANCA and a UC diagnosis (R²=0.82 and AUC=0.98%). The relative risk of non-response increased 15 fold when number of risk factors increased from 0-2 to ≥3. The combination of phenotype and genotype is most predictive of primary non response to anti-TNFα in pediatric IBD. Defining predictors of response to anti-TNFα will allow the identification of patients who will not benefit from this class of therapy.

### Example 27

### Final Predictive Model—Patient Population

A total of 94 pediatric CD and UC patients (age at diagnosis <21 years) followed at Cedars-Sinai Medical Center (CSMC) by one clinician (MD) were enrolled in this study. All subjects must have received at least 2 doses of (weeks 0 and 2) of infliximab to be eligible. Infliximab was chosen as the first line anti-TNF used in both CD and UC for children. This study was approved by the institutional IRB.

### Example 28

### Final Predictive Model—Phenotyping

All data was collected by chart review and stored in a secured database. For the purpose of this study phenotype was defined as all variables that were not genetic.

Clinical Phenotype: These included demographic and clinical variables: age, gender, IBD subtype (CD vs. UC), disease duration, age at diagnosis, age at initiation of infliximab, immunomodulator history, steroid history, Harvey Bradshaw Index (HBI) activity scores, Partial Mayo scores and reason for infliximab discontinuation.

Immune Phenotype: Serum was collected on all patients and analyzed at CSMC. Serum immune responses: anti-Saccharomyces cerevisiae antibodies (ASCA IgG and IgA), perinuclear anti-nuclear cytoplasmic antibody (pANCA), anti-flagellin (antiCBir1), anti-outer membrane porin C (anti-OmpC) and anti-Pseudomonas fluorescens-associated sequence I2 (anti-I2) were analyzed blinded to therapeutic responsiveness by ELISA as previously described.

### Example 29

### Final Predictive Model—Genotype

Genotyping was performed at Children's Hospital of Philadelphia (CHOP) using the Illumina Human550 platform (n=70) and the Medical Genetics Institute at CSMC using the Illumina Human610 platform for CD samples (n=17) and HumanCNV370 platform for UC samples (n=11) (23). First, genotype data were tested for association between previously reported IBD susceptibility SNPs and anti-TNFα response. Table 11 illustrates the 28 SNPs included in this part of the analysis and references the GWAS that first reported these associations with disease. Twenty-one SNPs from a previous CD meta-analysis GWAS, 5 SNPS from various UC GWAS and 2 SNPs from a pediatric IBD GWAS were analyzed. Second, the genome wide data were tested for association with anti-TNFα response (“pharmacogenetic” GWAS, see below).

TABLE 11
Known Genetic Susceptibility Loci

| | GWAS Reference |
|---|---|
| | 1 = Adult CD (14) |
| Chromosome/ | 2 = Adult UC (15-17) |
| Loci of interest | 3 = Pediatric IBD (18) |
| 1p13/PTPN22 | 1 |
| 1p36 | 2 |
| 1q23/ITLN1 | 1 |
| 1q24 | 1 |
| 1q21.2/ECM1 | 2 |
| 1q32.1/IL10 | 2 |
| 1p31/IL23R | 1 |
| 2q37/ATG16L1 | 1 |
| 3p21/MST1 | 1 |
| 5p13/PTGER4 | 1 |
| 5q31/IBD5 | 1 |
| 5q33/IL12b | 1 |
| 6p21/HLA- | 2 |
| DQA1/TNFα, TNFβ | |
| 6p22/CDKAL1 | 1 |
| 6q27/CCR6 | 1 |
| 8q24.13/TRIB1 | 1 |
| 9p24/JAK2 | 1 |
| 9q32/TNFSF15 | 1 |
| 10q21/ZNF365 | 1 |
| 10q24/NKX2-3 | 1 |
| 12q15 | 2 |
| 13q14/C13orf31 | 1 |
| 16q12/NOD2 | 1 |
| 17q12/CCL2 | 1 |
| 17q21/ORMDL3/STAT3 | 1 |
| 20q13/TNFRSR6B | 3 |
| 21q22/COSLG | 1 |
| 21q22.2/BRWD1 | 3 |

### Example 30

### Final Predictive Model—Outcomes and Definitions

The primary outcome of this study was to identify genetic loci associated with primary non-response defined as:
CD: Failure to decrease HBI ≥3 points (24) or increase from baseline at week 10 or 4 weeks after their last infusion if they did not receive the 3^{rd} induction dose
UC: Failure to decrease ≥2 points or increase from baseline in the sum of stool frequency and rectal bleeding subscores of the partial Mayo score (7) and no improvement in the physician's global assessment subscore at week 10 or 4 weeks after their last infusion if they did not receive the 3^{rd} induction dose

### Example 31

### Final Predictive Model—Univariate Analysis

Association between clinical and demographic variables and primary non-response: The Chi-square test was used to check the association of primary non-response with the following categorical variables: CD vs. UC, male vs. female, serum immune response positivity, percentage of immunomodulator use at the start of infliximab and primary non-response. The Student t test was used for associations of continuous variables; age of diagnosis, disease duration and duration of immunomodulator use at the start of infliximab.

Association between known IBD susceptibility loci and primary non-response: The Chi-square test was applied to test the association between each SNP (Table 11) and primary non-response. A dominant model based on the presence of the rare allele was assumed. Relative risk was calculated by comparing the risk of non-response in the patients with a specific genotype versus those without the genotype.

Pharmacogenetic genome wide association study (GWAS):
a. Principal components (PC) analysis (using Eigenstrat) was conducted to examine population stratification (25). All the subjects formed one cluster with no significant outliers. There was no need to correct for population stratification during the association analysis, as the first ten PC evaluated were not significantly associated with primary non-response.
b. For the purpose of quality control, SNPs with a minor allele frequency (MAF) <0.01, genotype failure rate >0.10, HWE P value <0.001 were excluded from the analysis. Allelic association between an individual SNP and primary non-response was carried out by chi-square test in PLINK (26). The first 10 SNPs with the most significant results were then retained for modeling. Following quality control, 301,742 SNPs were available in all data sets for analysis.

### Example 32

### Final Predictive Model—Multivariate Analysis

Predictive models of primary non-response: Models to predict non-response were built using step-wise multiple logistic regression, combining a) IBD susceptibility SNPs, b) the top 10 hits from the pharmacogenetic GWAS analysis, c) serology status and d) IBD subtypes. Since rs5975493 and rs7059861 are in high linkage disequilibrium, only rs7059861 was kept in the model. Exact logistic regression was used if the estimate from a regular regression model was not available. The significance level for a variable to enter and stay in the model was 10%. The likelihood based pseudo-R squared from the logistic regression model was used to measure the strength of association as well as the proportion of variance of the outcome accounted by the model's independent variables.

Models were built at five different levels:
I. demographic variables, serology status and IBD subtype only
II. known IBD susceptibility SNPs only
III. model I and II combined
IV. pharmacogenetic GWAS SNPs (dominant model assumed) only
V. model III and IV combined (final model). (known IBD susceptibility SNPs, pharmacogenetic GWAS top hits, serologic status and clinical variables)

Clinical Utility Measures: The area under the Receiver Operating Characteristic (ROC) curve (AUC) was used as a measure of the predictive performance of the final model. The risk score was calculated based on the final model by assigning each risk phenotype or genotype as 1 point. Sensitivity [# true positives/(# of true positives+# of false negatives)], specificity [# of true negatives/(# of true negatives+# of false positives)], accuracy [(sensitivity+specificity)/2] and positive likelihood ratio test (sensitivity/(1−specificity)) for primary non response were also calculated for the final model (28). All statistical analysis was conducted by SAS software v9.1 (SAS Institute; Cary, NC.).

### Example 33

### Final Predictive Model—Results of Patient Population and Phenotype Associations

Of the 94 patients evaluated, 22 patients (23%) met the criteria of primary non-response. Table 12 illustrates the key demographic data for both responders and non responders. A diagnosis of UC (p<0.0001) and pANCA positivity (p=0.0001) were associated with primary non-response. Gender, mean age at diagnosis, disease duration at initiation of infliximab, percentage of immunomodulator use and duration of use at start of infliximab did not differ between the two groups.

TABLE 12
Phenotype Associations with Therapeutic Outcomes to Anti-TNFα

| CLINICAL VARIABLE | NON- RESPONSE | RESPONSE | P Value |
|---|---|---|---|
| UC vs. CD | 14:8 | 6:66 | <0.0001 |
| Mean age at diagnosis | 10.1 | 10.5 | 0.71 |
| (years) | | | |
| Gender M:F | 8:14 | 40:32 | 0.12 |
| Disease duration at start | 26 | 25 | 0.96 |
| of IFX (months) | | | |
| IMM use at start | 79% | 92.8% | 0.1 |
| of IFX (%) | | | |
| Duration of IMM at start | 12.7 | 15.3 | 0.57 |
| of IFX (months) | | | |
| pANCA+ | 76.2% | 29% | 0.0001 |
| ASCA+ | 0% | 46.9% | 0.0003 |

### Example 34

### Final Predictive Model—Results of Genetic Associations: Univariate Analysis

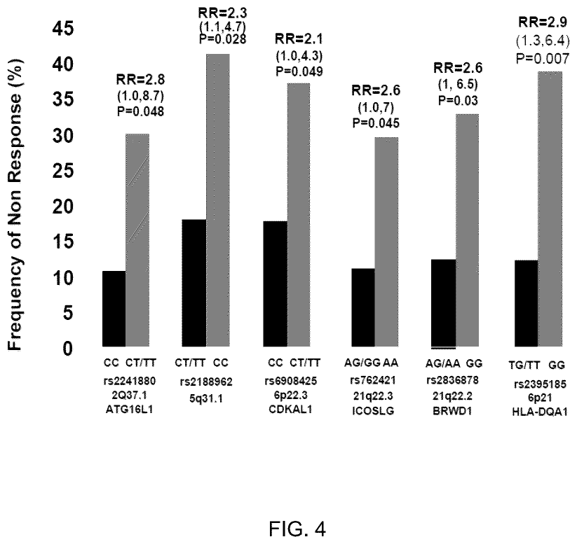

Known IBD Susceptibility Loci: Of the 28 previously identified genetic loci (Table 11), 6 were found to be significantly associated with primary non-response. FIG. 4 herein illustrates the frequency of primary non-response for the different genotypes of these 6 SNPs. Four of the 6 SNPs are from the CD meta-analysis, 1 from the UC GWAS and 1 from pediatric IBD GWAS. For this analysis, the dominant model of the rare allele was assumed. The common allelic variant was associated with non-response in 4 of the 6 SNPs. Table 13 compares the reported IBD risk allele with the allele found to be associated with non-response in this study for all 6 SNPs. In only 2 of the SNPs was the disease risk allele the same as that found to be associated with non response to anti-TNFα.

TABLE 13
Allelic Variants

| | GWAS Reference | | |
|---|---|---|---|
| | 1 = Adult CD (14) | | |
| SNP and | 2 = Adult UC (15-17) | IBD Risk | Non-Response |
| Gene/Locus | 3 = Pediatric IBD (18 | Allele | Allele |
| rs2241880 | 1 | C | T |
| 2q37/ATG16L1 | | | |
| rs2188962 | 1 | T | C |
| 5q31 | | | |
| rs6908425 | 1 | C | T |
| 6p22/CDKAL1 | | | |
| rs762421 | 1 | G | A |
| 21q22/ICOSLG | | | |
| rs2395185 | 2 | G | G |
| 6p21/HLA-DAQ1 | | | |
| rs2836878 | 3 | G | G |
| 21q22/BRWD1 | | | |

Pharmacogenetic GWAS: Table 14 lists the results of the chi square analyses for the pharmacogenetic GWAS. Only those SNPs with a p value <10⁻⁴ are listed.

TABLE 14
Pharmacocienetic GWAS: p < 0.0001

| | | | Gene | Other | | |
|---|---|---|---|---|---|---|
| Chromosome | SNP | Position* | db129*, | loci**, | OR | P |
| 13 | rs1155848 | 78786477 | | RBM26 | 35.73 | 5.35E−07 |
| 5 | rs1592749 | 159904599 | | | 5.949 | 1.09E−06 |
| X | rs765132 | 133150270 | | 347475 | 35 | 1.09E−06 |
| | | | | \|402425 | | |
| | | | | \|644403 | | |
| 6 | rs4707930 | 72463040 | | | 6.603 | 5.55E−06 |
| 10 | rs7905482 | 81853944 | | PLAC9 | 4.875 | 1.08E−05 |
| | | | | \|389988 | | |
| | | | | 642506 | | |
| | | | | \|642521 | | |
| | | | | \|642538 | | |
| | | | | \|727879 | | |
| 20 | rs6100556 | 57701043 | PHACTR3 | PHACTR3 | 4.951 | 1.23E−05 |
| X | rs7059861 | 133191565 | | 347475 | 8.333 | 1.62E−05 |
| | | | | \|402425 | | |
| X | rs5975453 | 133212999 | | 347475 | 8.333 | 1.62E−05 |
| | | | | 402425 | | |
| 10 | rs4077511 | 5568172 | | CALML3 | 8.214 | 1.74E−05 |
| | | | | CALML5 | | |
| | | | | 100132159 | | |
| 2 | rs975664 | 75222305 | TACR1 | TACR1 | 0.1667 | 1.75E−05 |
| 3 | rs4855535 | 69017124 | FAM19A4 | FAM19A4 | 5.619 | 1.77E−05 |
| 17 | rs4796606 | 36917613 | | KRT13 | 11.73 | 1.98E−05 |
| | | | | \|KRT15 | | |
| | | | | \|KRT32 | | |
| | | | | \|KRT35 | | |
| | | | | \|KRT38 | | |
| | | | | \|KRT37 | | |
| | | | | \|KRT36 | | |
| 8 | rs2943177 | 88121513 | CNBD1 | CNBD1 | 6.412 | 2.06E−05 |
| 8 | rs11991611 | 139676572 | COL22A1 | FAM135B | 15.29 | 2.12E−05 |
| | | | | COL22A1 | | |
| 10 | rs3740543 | 129140065 | DOCK1 | DOCK1 \| | 4.932 | 2.18E−05 |
| | | | | NPS | | |
| 21 | rs2825673 | 19888587 | | 100128057 | 6.438 | 2.28E−05 |
| 1 | rs7521532 | 70351206 | LRRC7 | SFRS11 | 7.143 | 2.54E−05 |
| | | | | \|LRRC7 | | |
| 3 | rs4605505 | 141323576 | CLSTN2 | CLSTN2 | 7.143 | 2.54E−05 |
| 8 | rs7003556 | 5213929 | | | 7.143 | 2.54E−05 |
| 14 | rs1243519 | 94370110 | | GSC | 6 | 3.06E−05 |
| 5 | rs2044111 | 22897160 | | CDH12 | 4.444 | 3.54E−05 |
| 6 | rs2103867 | 47374655 | TNFRSF21 | TNFRSF21 | 12.07 | 3.6E−05 |
| 7 | rs17168564 | 14977349 | | | 8.286 | 3.68E−05 |
| 6 | rs10485363 | 13245751 | PHACTR1 | PHACTR1 | 5.182 | 3.87E−05 |
| 5 | rs7726515 | 129736249 | | | 5.187 | 4E−05 |
| 11 | rs835780 | 44816947 | | TSPAN18 | 5.187 | 4E−05 |
| 11 | rs835791 | 44823513 | | TSPAN18 | 5.187 | 4E−05 |
| 1 | rs3795727 | 154856074 | HAPLN2 | GPATCH4 | 4.5 | 4.1E−05 |
| | | | | \|HAPLN2 | | |
| | | | | BCAN | | |
| | | | | IQGAP3 | | |
| | | | | 646129 | | |
| 6 | rs6906890 | 13069206 | PHACTR1 | PHACTR1 | 5.899 | 4.31E−05 |
| 11 | rs7124825 | 44812803 | | TSPAN18 | 0.1263 | 4.31E−05 |
| 19 | rs302827 | 61102034 | NLRP13 | NLRP13 | 4.545 | 4.45E−05 |
| | | | | NLRP8 | | |
| | | | | NLRP1 | | |
| 12 | rs2723829 | 11819364 | ETV6 | ETV6 | 4.333 | 4.47E−05 |
| 2 | rs1372256 | 141070866 | LRP1B | LRP1B | 4.626 | 4.65E−05 |
| 4 | rs13138970 | 155494510 | DCHS2 | DCHS2 | 5.297 | 4.75E−05 |
| 20 | rs1205434 | 36302930 | KIAA1755 | BPI \| TGM2 | 0.1526 | 4.75E−05 |
| | | | | KIAA1755 | | |
| 13 | rs9556658 | 96476252 | | OXGR1 | 6.111 | 4.92E−05 |
| 2 | rs7588326 | 75202554 | TACR1 | TACR1 | 0.1826 | 5.07E−05 |
| 2 | rs3771823 | 75205456 | TACR1 | TACR1 | 0.1826 | 5.07E−05 |
| 20 | rs1555901 | 20762132 | | | 0.1826 | 5.07E−05 |
| X | rs4465121 | 133249071 | | PHF6 | 7 | 5.26E−05 |
| 6 | rs12527937 | 16652564 | ATXN1 | ATXN1 | 5.5 | 5.74E−05 |
| 7 | rs10269232 | 10442565 | | 100128638 | 6.368 | 6.33E−05 |
| 6 | rs3757105 | 73950398 | KCNQ5 | KCNQ5 | 24.2 | 6.51E−05 |
| 18 | rs1667216 | 27383929 | | DSG2 \| TTR | 4.952 | 6.75E−05 |
| 12 | rs278917 | 39666755 | CNTN1 | CNTN1 | 4.172 | 6.93E−05 |
| 6 | rs9404502 | 104605814 | | 100129694 | 5.409 | 7.01E−05 |
| X | rs5977968 | 133151511 | | 347475 | 7.333 | 7.03E−05 |
| | | | | 402425 | | |
| | | | | 644403 | | |
| 1 | rs12567958 | 154861280 | HAPLN2 | GPATCH4 | 4.167 | 7.03E−05 |
| | | | | HAPLN2 | | |
| | | | | BCAN | | |
| | | | | IQGAP3 | | |
| | | | | 646129 | | |
| 8 | rs1880473 | 88079222 | CNBD1 | CNBD1 | 5.085 | 7.09E−05 |
| 4 | rs7689941 | 182246739 | hCG_2025798 | 728081 | 4.244 | 7.3E−05 |
| 17 | rs12937472 | 15194290 | | PMP22 | 8.509 | 7.5E−05 |
| | | | | TEKT3 | | |
| 5 | rs4301261 | 159902033 | | | 5.433 | 7.59E−05 |
| X | rs1264379 | 132893432 | GPC3 | GPC3 | 6.092 | 7.59E−05 |
| X | rs6529954 | 4194349 | | | 0.1781 | 7.69E−05 |
| 22 | rs3088103 | 25250048 | TPST2 | CRYBA4 | 4.308 | 7.77E−05 |
| | | | | \|TPST2 | | |
| | | | | TFIP11 | | |
| | | | | HPS4 | | |
| | | | | 644380 | | |
| | | | | 653715 | | |
| | | | | 729905 | | |
| | | | | 100128401 | | |
| | | | | HMGB1L10 | | |
| 6 | rs4711716 | 42375201 | TRERF1 | TRERF1 | 8.727 | 7.82E−05 |
| | | | | 387535 | | |
| | | | | 653802 | | |
| 7 | rs10464448 | 141433796 | MGAM | MGAM | 0.134 | 8.32E−05 |
| X | rs12559781 | 4225952 | | | 7.973 | 8.49E−05 |
| 7 | rs2540678 | 36227249 | EEPD1 | EEPD1 | 4.6 | 8.54E−05 |
| 4 | rs7659755 | 182246126 | hCG_2025798 | 728081 | 4.461 | 8.62E−05 |
| 13 | rs770389 | 50043157 | LOC730194 | 730194 | 5.1 | 8.96E−05 |
| 21 | rs2825699 | 19934102 | | | 5.1 | 8.96E−05 |
| 12 | rs7309734 | 39564210 | CNTN1 | CNTN1 | 4.171 | 9.7E−05 |
| 2 | rs11903032 | 3840992 | | | 5.469 | 9.78E−05 |
| 8 | rs10808755 | 68679235 | CPA6 | CPA6 | 0.08701 | 9.87E−05 |

*Defined using dbSNP Build 129
**Loci include genes that overlap each SNP within 100 kb of 5′ end and 10 kb of 3′ end of each gene. Numbers refer to dbGene (NCBI).

### Example 36

### Final Predictive Model—Results of Multivariate Analysis

Predictive models of non-response: Logistic multiple regression was employed to develop models of primary non-response. Five different models were developed. Model 1 examined the significance of pANCA and IBD subtype (UC vs. CD). Both PANCA (OR 5.4; p=0.01) and the diagnosis of UC (OR 15.0; p=0.0001) remained significant in model I, with an R squared (R²) of 0.48. Model II (R²=0.30) examined the 6 SNPs from the univariate analysis. Four (4) of the 6 IBD susceptibility SNPs remained significant; rs2188962 (5q31) (OR 3.3; p=0.04), rs6908425 (6p22/CDKAL1) (OR 3.2; p=0.04), rs2836878 (21q22/BRWD1) (OR 3.3; p=0.05) and rs2395185 (6p21/HLA-DAQ1) (OR 4.6; p=0.01). Table 15 shows the results of Model Ill which included serology, IBD subtype and susceptibility SNPs (combining the variables of models I and II). 3 SNPs survived the model when combined with these other independent variables. Model IV analyzed the top 10 SNPs from the pharmacogenetic GWAS and only 4 SNPs remained significant; rs975664 (TACR1) (OR 17.6, p=0.0006), rs4855535 (FAM19A4) (OR 8.8, p=0.006), rs4796606 (KRT32 KRT35 KRT36 KRT13) (OR 13.4, p=0.01) and rs765132 (OR 30.1, p=0.03). The R squared for this particular model was 0.67. The results of the final model (V) are shown in Table 16. Model V (R²=0.82, including all variables) examined the associations of pANCA, diagnosis of UC, the 6 known susceptibility SNPs and the top 10 SNPs from the pharmacogenetic GWAS. UC, PANCA, 3 SNPs from the pharmacogenetic GWAS, and rs2836878 (21q22/BRWD1), a susceptibility SNP remained significant.

TABLE 15
Model III: susceptibility SNPs and phenotype

| Model | Variable | P value | OR | R squared |
|---|---|---|---|---|
| III: | UC vs. CD | 0.0008 | 14.7 | 0.59 |
| | pANCA pos | 0.004 | 15.3 | |
| | rs6908425 | 0.05 | 4.6 | |
| | 6p22.3 | | | |
| | CDKA1 | | | |
| | rs2836878 | 0.02 | 9.8 | |
| | 21q22.2 BRWD1 | | | |
| | rs2395185 | 0.047 | 5.4 | |
| | 6p21 | | | |
| | HLA-DQA1 | | | |

TABLE 16
Model V: Final Model

| Model | Variable | P value | OR | R squared |
|---|---|---|---|---|
| V: | Diagnosis: UC vs. CD | 0.008 | 28.9 | 0.82 |
| | pANCA pos vs neg | 0.03 | 15.4 | |
| | rs975664 | 0.01 | 26.5 | |
| | 2p12/TACR1 | | | |
| | rs4855535 | 0.02 | 10.8 | |
| | 3p14/FAM19A4 | | | |
| | rs6100556 | 0.02 | 13.8 | |
| | 20q13/PHACTR3 | | | |
| | rs2836878 | 0.07 | 8.0 | |
| | 21q22/BRWD1 | | | |

### Example 37

### Final Predictive Model—Results of Clinical Utility Measures

The potential clinical utility of the final model (V) was calculated. Table 17 lists the AUC, sensitivity, specificity, accuracy and positive likelihood ratio of non response in a patient who had at least 3 of the 6 risk factors of non response based on model V. In addition, the negative likelihood ratio, i.e. the likelihood that a patient will not be a non-responder if at least 3 risk factors are absent, was calculated at 0.06.

TABLE 17
Clinical Utility Measures

| | | | | | | Positive Likelihood |
|---|---|---|---|---|---|---|
| Model | Variable | AUC | Sensitivity | Specificity | Accuracy | Ratio |
| V: | Diagnosis | 0.98 | 0.95 | 0.88 | 0.92 | 8 |
| | pANCA | | | | | |
| | rs975664 2p12 | | | | | |
| | TACR1 | | | | | |
| | rs4855535 | | | | | |
| | 3p14 FAM19A4 | | | | | |
| | rs6100556 | | | | | |
| | 20q13 | | | | | |
| | PHACTR3 | | | | | |
| | rs2836878 | | | | | |
| | 21q22 BRWD1 | | | | | |

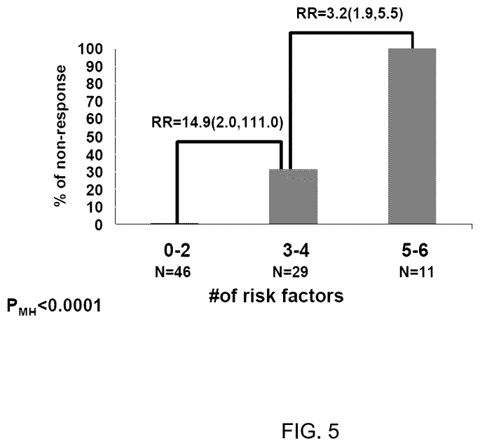

The relative risk of non-response was calculated based on the number of risk factors (model V) carried by an individual patient. Both the frequency of non-response and the relative risk increase with increasing number of risk factors (p<0.0001) (FIG. 5).

### Example 38

### Final Predictive Model

TABLE 18

| MODEL | MODEL DESCRIPTOR | R SQUARED | AUC |
|---|---|---|---|
| I | Diagnosis and pANCA | 0.48 | 0.9 |
| II | Known Susceptibility SNPs only | 0.3 | 0.8 |
| III | Diagnosis, pANCA and known | 0.59 | 0.93 |
| | susceptibility SNPs | | |
| IV | Pharmacogenetic GWAS SNPS only | 0.67 | 0.94 |
| V | Diagnosis, pANCA, known | 0.82 | 0.98 |
| | susceptibility SNPs AND | | |
| | pharmacogenetic GWAS SNPs | | |

Anti-TNFα is an important and effective class of therapies for the management of both adult and pediatric IBD patients. Clinical experience suggests inter-individual variation in efficacy, both induction and maintenance, and in the occurrence of side effects. There are likely multiple host factors that influence these variations such as disease and immune phenotype as well as genetic background. The ability to predict which patient would have a lower likelihood of response before treatment is initiated in order to minimize exposure to potentially ineffective therapies may be an important consideration in IBD patients. In the current era of risk/benefit balance, this concept may be very timely.

As described herein, the inventors tested the associations of known and novel genetic loci with primary response outcome and developed a predictive model of primary non response using clinical phenotype, serologic and genetic variables. Six of the 28 known susceptibility loci tested were found to be associated with primary non response in the univariate analysis. The relative risk of primary non response ranged from 2.1-2.9. When tested in the multivariate analysis, 4 of these loci remained significant. However when combined with the novel pharmacogenetic GWAS loci, only 1 loci remained significant. This one locus was initially reported in the pediatric IBD GWAS reported by Kugathasan et al. The functional significance of this locus remains unknown. Of the 10 novel pharmacogenetic GWAS loci tested in the predictive model, 3 remained significant in the final model. TACR1 is a receptor for substance P a known pro-inflammatory molecule. PHACTR3 (phosphatase and actin regulator 3) is associated with the nuclear scaffold in proliferating cells. While there is little known about FAM19A4 it is thought to be structurally related to MIP1a and function as a chemokine. The pharmacogenetic GWAS identified top loci did substantially improve the strength of the prediction of non-response compared to known susceptibility loci. In addition, a diagnosis of UC and PANCA positivity was independently associated with primary non-response. Table 18 compares the r-squared and AUC values for all 5 models. The combination of genotype, phenotype and serotype was the best predictive model of non response to anti-TNFα with an r-squared of 0.82 and an AUC of 0.98, and substantially better than the models that included only known IBD SNPs. (models II or III).

The findings suggest that the majority of the known IBD susceptibility loci do not appear to greatly modify or influence primary response outcomes to anti-TNFα in pediatric IBD patients. This raises the possibility that the majority of genes that are associated with risk of disease may not influence the immune pathways that should be targeted to control or modify disease activity. The results of previously reported candidate gene association studies with anti-TNFα response have not been translated into the clinic and the functional significance of the genes tested remain unknown (8-13). The functionality of the cytokines and/or receptor renders them of interest as it relates to therapeutic outcome. With the GWAS approach, however, no a priori assumptions need to be set, as there is not a prior focus on a particular protein or target or enzyme as it relates to drug response. This hypothesis generating approach allows the identification of genetic variants that are associated with response and non-response and thus potentially identify pathways that are responsible and may well be apparent from a functional and mechanistic perspective. In this study the inventors have analyzed the associations with primary non-response only, as believed this was of the greatest clinical relevance.

As described herein, the inventors have included all pediatric IBD subjects receiving anti-TNFα therapy. Currently clinicians treat both CD and UC with anti-TNF therapy and the clinical trial data suggest similar primary non response and steroid free remission outcomes for both disease subtypes. Moreover there is genetic and serologic evidence that there is pathway biology overlap within the spectrum of CD and UC phenotype. The inventors analyzed UC, CD and shared susceptibility loci and the pharmacogenetic GWAS would identify loci that are independent of disease phenotype.

Defining predictors of response to anti-TNFα will assist clinicians in choosing the appropriate therapy for the appropriate IBD patient, with the goal of maximizing efficacy and minimizing toxicity. As research progresses in defining the characteristics of patients who require biologics, of equal importance will be the research as proposed herein to individualize therapy based on who will or will not respond to different classes of IBD therapeutic interventions. The development of adverse events to anti-TNF therapies such as lymphoma, and sepsis naturally induce caution in clinicians who would like the ability to appropriately select patients who are most likely to respond to these therapies.

As readily apparent to one of skill in the art, after a diagnosis of nonresponsiveness to anti TNFα in an individual, the invention also includes the administration of any number of treatments that may act as an alternative to anti TNFα therapy, such as natalizumab for example. Similarly, after a diagnosis of responsiveness to anti TNFα therapy, any number of examples of anti TNFα therapy may be used, such as infliximab or cyclosporin. Additionally, as apparent to one of skill in the art, the various embodiments described herein may be used in conjunction with any number of additional inflammatory bowel disease treatments, therapies and methods of diagnosis and prognosis. Finally, as apparent to one of skill in the art, the invention may be applied to any number of conditions and diseases related to or potentially affected by anti TNFα therapy and the invention is not limited to inflammatory bowel disease. While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Accordingly, the invention is not limited except as by the appended claims.

### REFERENCES

1. Beaugerie L, Seksik P, Nion-Larmurier I, Gendre J P, Cosnes J. Predictors of Crohn's disease. Gastroenterology. 2006; 130:650-656
2. Van Limbergen J. Russell R K. Drummond H E. Aldhous M C. Round N K. Nimmo E R. Smith L. Gillett P M. McGrogan P. Weaver L T. Bisset W M. Mahdi G. Arnott I D. Satsangi J. Wilson D C. Definition of phenotypic characteristics of childhood-onset inflammatory bowel disease. Gastroenterology 2008; 135:1114-22
3. Hyams J. Crandall W. Kugathasan S. Griffiths A. Olson A. Johanns J. Liu G. Travers S. Heuschkel R. Markowitz J. Cohen S. Winter H. Veereman-Wauters G. Ferry G. Baldassano R. REACH Study Group. Induction and maintenance infliximab therapy for the treatment of moderate-to-severe Crohn's disease in children. Gastroenterology 2007; 132:863-73
4. Hanauer S B, Feagan B G, Lichtenstein G R, Mayer L F, Schreiber S, Colombel J F, Rachmilewitz D, Wolf D C, Olson A, Bao W, Rutgeerts P; ACCENT I Study Group. Maintenance infliximab for Crohn's disease: the ACCENT I randomized trial. Lancet 2002; 359:1541-1549.
5. Colombel J F. Sandborn W J. Rutgeerts P. Enns R. Hanauer S B. Panaccione R. Schreiber S. Byczkowski D. Li J. Kent J D. Pollack P F. Adalimumab for maintenance of clinical response and remission in patients with Crohn's disease: the CHARM trial. Gastroenterology 2007; 132:52-65.
6. Schreiber S. Khaliq-Kareemi M. Lawrance I C. Thomsen O O. Hanauer S B. McColm J. Bloomfield R. Sandborn W J. PRECISE 2 Study Investigators. Maintenance therapy with certolizumab pegol for Crohn's disease. N Engl J Med. 2007; 357:239-250.
7. Rutgeerts P. Sandborn W J. Feagan B G. Reinisch W. Olson A. Johanns J. Travers S. Rachmilewitz D. Hanauer S B. Lichtenstein G R. de Villiers W J. Present D. Sands B E. Colombel J F. Infliximab for induction and maintenance therapy for ulcerative colitis. New England Journal of Medicine 2005; 353:2462-76
8. Pierik M. Vermeire S. Steen K V. Joossens S. Claessens G. Vlietinck R. Rutgeerts P. Tumour necrosis factor—a receptor 1 and 2 polymorphisms in inflammatory bowel disease and their association with response to infliximab. Aliment. Pharmacol. Ther 2004; 20:303-310
9. Mascheretti S. Hampe J. Kuhbacher T. Herfarth H. Krawczak M. Folsch U R. Schreiber S. Pharmacogenetic investigation of the TNF/TNF-receptor system in patients with chronic active Crohn's disease treated with infliximab. Pharmacogenomics 2002; J. 2:127-136
10. Louis E. Vermeire S. Rutgeerts P. De Vos M. Van Gossum A. Pescatore P. Fiasse R. Pelckmans P. Reynaert H. D'Haens G. Malaise M. Belaiche J. A positive response to infliximab in Crohn disease: association with a higher systemic inflammation before treatment but not with _308 TNF gene polymorphism. Scand. J. Gastroenterol. 2002; 37:818-824
11. Taylor K D. Plevy S E. Yang H. Landers C J. Barry M J. Rotter J I. Targan S R. LTa 1-1-1-1 haplotype is associated with negative response in Crohn's disease ANCA pattern and LTA haplotype relationship to clinical responses to anti-TNF antibody treatment in Crohn's disease. Gastroenterology 2001; 120, 1347-1355.
12. Vermeire S. Louis E. Rutgeerts P. De Vos M. Van Gossum A. Belaiche J. Pescatore P. Fiasse R. Pelckmans P. Vlietinck R. Merlin F. Zouali H. Thomas G. Colombel J F. Hugot JP.NOD2/CARD15 does not influence response to infliximabin Crohn's disease. Gastroenterology 2002; 123, 106-111.
13. Urcelay E. Mendoza J L. Martinez A. Fernandez L. Taxonera C. Diaz-Rubio M. de la Concha E G. IBD 5 (5q31) T T is associated with negative response in Crohn's disease. IBD5 polymorphisms in inflammatory bowel disease: association with response to infliximab. World J. Gastroenterol 2005; 11:1187-1192
14. Barrett J C. Hansoul S. Nicolae D L. Cho J H. Duerr R H. Rioux J D. Brant S R. Silverberg M S. Taylor K D. Barmada M M. Bitton A. Dassopoulos T. Datta L W. Green T. Griffiths A M. Kistner E O. Murtha M T. Regueiro M D. Rotter J I. Schumm L P. Steinhart A H. Targan S R. Xavier R J. NIDDK IBD Genetics Consortium. Libioulle C. Sandor C. Lathrop M. Belaiche J. Dewit O. Gut I. Heath S. Laukens D. Mni M. Rutgeerts P. Van Gossum A. Zelenika D. Franchimont D. Hugot J P. de Vos M. Vermeire S. Louis E. Belgian-French IBD Consortium. Wellcome Trust Case Control Consortium. Cardon L R. Anderson C A. Drummond H. Nimmo E. Ahmad T. Prescott N J. Onnie C M. Fisher S A. Marchini J. Ghori J. Bumpstead S. Gwilliam R. Tremelling M. Deloukas P. Mansfield J. Jewell D. Satsangi J. Mathew C G. Parkes M. Georges M. Daly M J. Genome-wide association defines more than 30 distinct susceptibility loci for Crohn's disease. Nat Genet 2008; 40:955-62
15. Franke A, Balschun T, Karlsen T H, Sventoraityte J, Nikolaus S, Mayr G, Domingues F S, Albrecht M, Nothnagel M, Ellinghaus D, Sina C, Onnie C M, Weersma R K, Stokkers P C, Wijmenga C, Maria Gazouli M, Strachan D, McArdle W L, Vermeire S, Rutgeerts P, Rosenstiel P, Krawczak M, Vatn M H, the IBSEN study group, Mathew C G, Schreiber Sequence variants in IL10, ARPC2 and multiple other loci contribute to ulcerative colitis susceptibility. Nat Genetics 2008; 40:710-712
16. Fisher S A. Tremelling M. Anderson C A. Gwilliam R. Bumpstead S. Prescott N J. Nimmo E R. Massey D. Berzuini C. Johnson C. Barrett J C. Cummings F R. Drummond H. Lees C W. Onnie C M. Hanson C E. Blaszczyk K. Inouye M. Ewels P. Ravindrarajah R. Keniry A. Hunt S. Carter M. Watkins N. Ouwehand W. Lewis C M. Cardon L. Welcome Trust Case Control Consortium. Lobo A. Forbes A. Sanderson J. Jewell D P. Mansfield J C. Deloukas P. Mathew C G. Parkes M. Satsangi J. Genetic determinants of ulcerative colitis include the ECM1 locus and five loci implicated in Crohn's disease. Nat. Genet 2008; 40:710-712.
17. Silverberg M S. Cho J H. Rioux J D. McGovern D P. Wu J. Annese V. Achkar J P. Goyette P. Scott R. Xu W. Barmada M M. Klei L. Daly M J. Abraham C. Bayless T M. Bossa F. Griffiths A M. Ippoliti A F. Lahaie R G. Latiano A. Pare P. Proctor D D. Regueiro M D. Steinhart A H. Targan S R. Schumm L P. Kistner E O. Lee A T. Gregersen P K. Rotter J I. Brant S R. Taylor K D. Roeder K. Duerr R H. Ulcerative colitis-risk loci on chromosomes 1p36 and 12q15 found by genome-wide association study. Nat Genetics. 41 (2): 216-20, 2009
18. Kugathasan S, Baldassano R N, Bradfield J P, Sleiman P M, Imielinski M, Guthery S L, Cucchiara S, Kim C E, Frackelton E C, Annaiah K, Glessner J T, Santa E, Willson T, Eckert A W, Bonkowski E, Shaner J L, Smith R M, Otieno F G, Peterson N, Abrams D J, Chiavacci R M, Grundmeier R, Mamula P, Tomer G, Piccoli D A, Monos D S Annese V, Denson L A, Grant S F, Hakonarson H. Loci on 20q13 and 21q22 are associated with pediatric-onset inflammatory bowel disease. Nat Genetics; 40; 1211-1215.
19. Ferrante M. Vermeire S. Katsanos K H. Noman M. Van Assche G. Schnitzler F. Arijs I. De Hertogh G. Hoffman I. Geboes J K. Rutgeerts P Predictors of early response to infliximab in patients with ulcerative colitis. Inflammatory Bowel Diseases 2007; 13:123-8
20. Vasiliauskas E A, Plevy S E, Landers C J, Binder S W, Ferguson D M, Yang H, Rotter J I, Vidrich A, Targan S R. Perinuclear antineutrophil cytoplasmic antibodies in patients with Crohn's disease define a clinical subgroup. Gastroenterology 1996; 110:1810-1819.
21. Anderson C A. Massey D C. Barrett J C. Prescott N J. Tremelling M. Fisher S A. Gwilliam R. Jacob J. Nimmo E R. Drummond H. Lees C W. Onnie C M. Hanson C. Blaszczyk K. Ravindrarajah R. Hunt S. Varma D. Hammond N. Lewis G. Attlesey H. Watkins N. Ouwehand W. Strachan D. McArdle W. Lewis C M. Wellcome Trust Case Control Consortium. Lobo A. Sanderson J. Jewell D P. Deloukas P. Mansfield J C. Mathew C G. Satsangi J. Parkes M. Investigation of Crohn's disease risk loci in ulcerative colitis further defines their molecular relationship. Gastroenterology 2009; 136:523-9.
22. Dubinsky M C, Kugathasan S, Mei L, Picornell Y, Nebel J, Wrobel I, Quiros A, Silber G, Wahbeh G Katzir L, Vasiliauskas E, Bahar R, Otley A, Mack D, Evans J, Rosh J, Oliva Hemker M, Leleiko L, Crandall W, Langton C, Landers C, Taylor K D, Targan S R, Rotter J I, Markowitz J, Hyams J for the Western Regional Pediatric IBD Research Alliance, Pediatric IBD Collaborative Research Group and the Wisconsin Pediatric IBD Alliance. Increased immune reactivity predicts aggressive complicating Crohn's disease in children. Clinical Gastroenterology and Hepatology 2008:6; 1105-11
23. A genome-wide scalable SNP genotyping assay using microarray technology. Gunderson K L, Steemers F J, Lee G, Mendoza L G, Chee M S. Nat Genet. 2005; 37:549-54
24. Harvey R F, Bradshaw J M. A simple Index of Crohn's disease activity. Lancet 1980; 1:514
25. Price A L, Patterson N J, Plenge R M, Weinblatt M E, Shadick N A, Reich D. Principal components analysis corrects for stratification in genome-wide association studies. Nat Genet. 2006 August; 38 (8): 904-9.
26. Purcell S, Neale B, Todd-Brown K, et al. PLINK: a tool set for whole-genome association and population-based linkage analyses. Am J Hum Genet 2007; 81:559-75.
27. Stokes, M E, Davis C S, Koch, G G. Categorical data analysis using the SAS system. 2nd edition. SAS press, 2000, Cary, NC, USA
28. Armitage P, Berry G, Matthews J N S. Statistical Methods in Medical Research. Fourth Edition. Wiley-Blackwell, Malden, MA, 2002
29. Yamazaki K. McGovern D. Ragoussis J. Paolucci M. Butler H. Jewell D. Cardon L. Takazoe M. Tanaka T. Ichimori T. Saito S. Sekine A. Iida A. Takahashi A. Tsunoda T. Lathrop M. Nakamura Y. Single nucleotide polymorphisms in TNFSF15 confer susceptibility to Crohn's disease. Human Molecular Genetics 2005. 14:3499-506
30. SLCO1B1 variants and statin-induced myopathy—a genomewide study. SEARCH Collaborative Group. Link E. Parish S. Armitage J. Bowman L. Heath S. Matsuda F. Gut I. Lathrop M. Collins R. New England Journal of Medicine 2008; 359:789-99
31. Burgner D. Davila S. Breunis W B. Ng S B. Li Y. Bonnard C. Ling L. Wright V J. Thalamuthu A. Odam M. Shimizu C. Burns J C. Levin M. Kuijpers T W. Hibberd M L. International Kawasaki Disease Genetics Consortium. A genome-wide association study identifies novel and functionally related susceptibility Loci for Kawasaki disease PLOS Genetics 2009; 5:e1000319.

1.-7. (canceled)

8. A method for hybridizing a genetic risk factor to determine whether a subject diagnosed with an inflammatory bowel disease is predicted to suffer from non-response to an anti-tumor necrosis factor (TNF) therapy, the method comprising:
hybridizing a risk allele-specific oligonucleotide to a sequence of the genetic risk factor, the genetic risk factor comprising a risk allele in a biological sample obtained from the subject diagnosed with the inflammatory bowel disease, wherein the risk allele comprises a “T” at nucleoposition 501 within rs666595, or a “T” at nucleoposition 2001 within rs35693; and
detecting binding of the risk allele-specific oligonucleotide and the sequence of the genetic risk factor, wherein the presence of the genetic risk factor in the biological sample as determined by the detected binding is predictive of non-response to the anti-TNF therapy.

9. The method of claim 8, wherein the risk allele further comprises a “C” at nucleoposition 501 within rs598672.

10. The method of claim 8, wherein the non-response to the anti-TNF therapy comprises primary non-response or secondary loss of response.

11. The method of claim 8, wherein the risk allele-specific oligonucleotide comprises a detectable probe comprising a reporter dye and a quencher molecule.

12. The method of claim 8, wherein the anti-TNF therapy comprises infliximab, or cyclosporine.

13. The method of claim 8, wherein the inflammatory bowel disease comprises Crohn's disease (CD), or ulcerative colitis (UC).

14. A method for screening a human genome for a genotype conferring a risk of non-response to an anti-tumor necrosis factor (TNF) therapy, the method comprising utilizing a nucleic acid amplification assay to detect a presence of one or more genetic risk factors in a biological sample obtained from a subject diagnosed with an inflammatory bowel disease (IBD), wherein the one or more genetic risk factors comprises a risk allele “T” at nucleoposition 501 within rs666595, or a risk allele “T” at nucleoposition 2001 within rs35693, wherein the presence of the one or more genetic risk factors is indicative of the risk of non-response to the anti-TNF therapy.

15. The method of claim 14, wherein the one or more genetic risk factors further comprises a risk allele “C” at nucleoposition 501 within rs598672.

16. The method of claim 14, wherein the non-response to the anti-TNF therapy comprises primary non-response or secondary loss of response.

17. The method of claim 14, wherein the inflammatory bowel disease comprises Crohn's disease (CD), or ulcerative colitis (UC).

18. The method of claim 14, wherein the nucleic acid amplification assay comprises polymerase chain reaction (PCR), quantitative PCR (qPCR), an allelic discrimination assay, or a genotyping assay.

19. The method of claim 14, further comprising treating the subject with an active agent that does not target anti-TNF if the genotype conferring the risk of non-response to the anti-TNF therapy is detected in the biological sample obtained from the subject.

20. The method of claim 15, wherein the one or more genetic risk factors comprises the risk allele “T” at nucleoposition 501 within rs666595, the risk allele “T” at nucleoposition 2001 within rs35693, and the risk allele “C” at nucleoposition 501 within rs598672.
